(19) United States
(12) Patent Application Publication
(10) Pub. No.: US 2026/0253682 A1
(43) Pub. Date: Aug. 27, 2026

(54) SYSTEM

(71) Applicant: SoftBank Group Corp., Tokyo (JP)

(72) Inventor: Tomoya IWAKAMI, Tokyo (JP)

(73) Assignee: SoftBank Group Corp., Tokyo (JP)

(21) Appl. No.: 19/536,258

(22) Filed: Feb. 11, 2026

(30) Foreign Application Priority Data
Feb. 21, 2025 (JP) 2025-026992

(51) Int. Cl. G16H 10/20; G16H 10/60; G16H 50/70

(52) U.S. Cl. CPC G16H 10/20; G16H 10/60; G16H 50/70

(57) ABSTRACT

The system according to the embodiment comprises a reception unit, an analysis unit, a determination unit, and a proposal unit. The reception unit inputs symptoms. The analysis unit analyzes the symptoms input by the reception unit. The determination unit determines an optimal consultation date and time based on the symptoms analyzed by the analysis unit. The proposal unit proposes an online diagnosis based on the consultation date and time determined by the determination unit.

### CROSS-REFERENCE TO RELATED APPLICATION

The present application claims priority to and incorporates by reference the entire contents of Japanese Patent Application No. 2025-026992 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that waiting times at hospitals are long, making it difficult to conduct efficient consultations for both patients and hospitals.

### SUMMARY OF THE INVENTION

The system according to the embodiment comprises a reception unit, an analysis unit, a determination unit, and a proposal unit. The reception unit inputs symptoms. The analysis unit analyzes the symptoms input by the reception unit. The determination unit determines an optimal consultation date and time based on the symptoms analyzed by the analysis unit. The proposal unit proposes an online diagnosis based on the consultation date and time determined by the determination unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

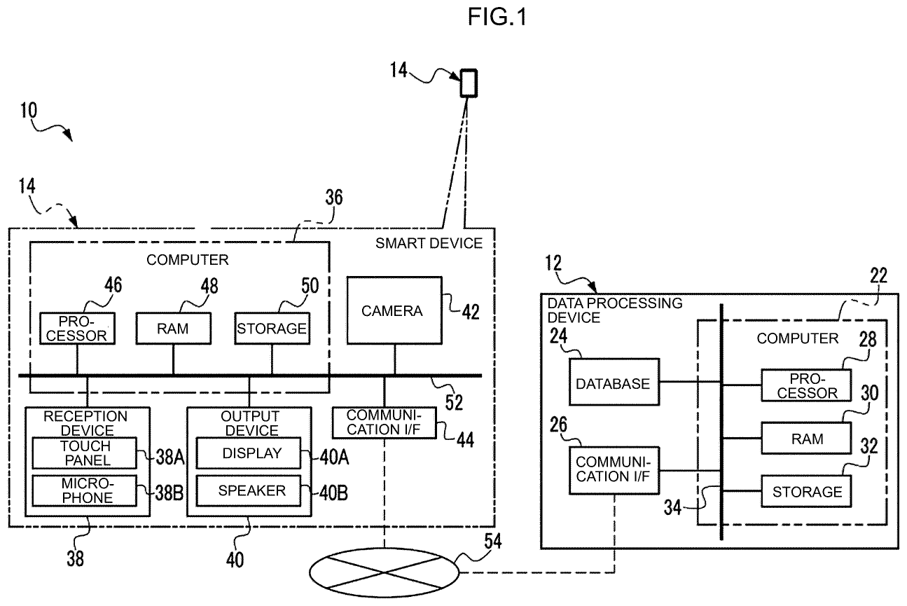

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

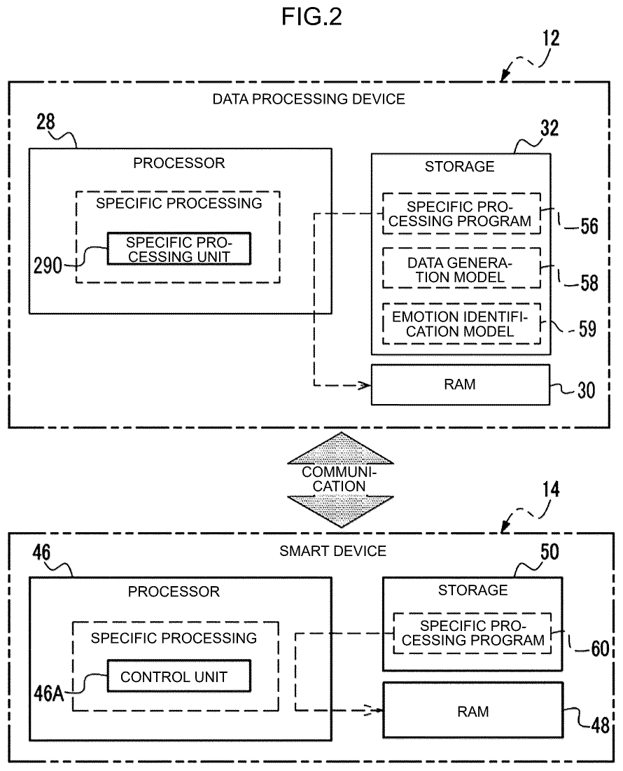

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The assistant system according to the embodiment of the present invention is a system for shortening waiting times at hospitals. This assistant system analyzes the symptoms of a patient using a generative AI and determines the optimal consultation date and time. When a patient visits the hospital, the symptoms are input. This information is input to the generative AI. The generative AI analyzes the input symptoms and, in cooperation with an image analysis AI, narrows down the patient's symptoms to a certain extent. Based on the narrowed-down symptoms, the system determines the optimal consultation date and time by considering existing reservations and the desired date and time of the patient. Furthermore, depending on the diagnosis result by the generative AI, the system can switch to online diagnosis, enabling more efficient diagnosis. For example, when a patient visits the hospital, inputs symptoms, and the generative AI analyzes the symptoms and proposes the optimal consultation date and time, the waiting time is shortened. In addition, by switching to online diagnosis, the patient does not need to go to the hospital and can receive consultation efficiently. As a result, the assistant system can shorten waiting times at hospitals and reduce the burden on patients. Moreover, hospitals can also conduct consultations more efficiently, thereby improving the overall efficiency of medical care. Specifically, the assistant system is composed of multiple computer modules such as a reception unit, analysis unit, determination unit, and proposal unit. The reception unit is designed so that patients can input symptoms in natural language or by selection via a user interface such as a touch panel terminal or smartphone application. The input data supports various formats, including text data (e.g., Japanese sentences such as “I have a headache,” “I have a cough,” up to 256 tokens), image data (facial photos or affected area images, RGB image tensor: 224×224×3), and audio data (audio files, 16 kHz, 16 bit, WAV format). These data are preprocessed by a preprocessing unit, which performs noise removal, normalization, tokenization, image resizing, etc., and are then input to the generative AI (for example, a transformer-based large language model or multimodal model). The generative AI vectorizes the input symptom text and image features using multi-layer self-attention mechanisms and convolutional neural networks, and performs symptom classification tasks (e.g., ICD-10 code classification, severity scoring) and urgency estimation tasks (e.g., probability value from 0 to 1, judged as urgent if threshold is 0.7 or higher). The image analysis AI uses CNN architectures such as ResNet or EfficientNet to analyze complexion (e.g., RGB histogram, blood color estimation) and facial expression (e.g., facial muscle feature point extraction, emotion estimation), and feeds back auxiliary features for urgency and severity to the generative AI. The analysis unit integrates these multimodal features and narrows down the symptoms (e.g., outputs the top three candidate diseases in a list, probability distribution for each disease). The determination unit cross-references the hospital's reservation management database (e.g., SQL table, reservation ID, date and time, attending physician, availability, etc.) and the patient's desired date and time (e.g., calendar format, desired date list), and calculates the optimal consultation date and time using a combinatorial optimization algorithm (e.g., constraint satisfaction problem, priority-based scheduling). The output is presented to the patient as a list of candidate dates and times (e.g., 2024/07/01 10:00, 2024/07/01 11:30, etc.) and recommendation scores (0 to 1). Furthermore, the proposal unit, based on the diagnosis result by the generative AI (e.g., labels of mild, moderate, or severe, online diagnosis availability flag), proposes switching to online diagnosis (e.g., generates video call links, automatically generates online diagnosis reservation forms). Examples of AI output include “Input: ‘I have a headache’+facial image→Output: ‘Urgency 0.2, candidate diseases: migraine (0.7), tension-type headache (0.2), cerebral hemorrhage (0.1)’” and “Input: ‘I have a cough’+facial image→Output: ‘Urgency 0.8, candidate diseases: pneumonia (0.6), bronchitis (0.3), common cold (0.1)’”. In subsequent processing, if urgency is high, immediate consultation slots are preferentially allocated, and if low, online diagnosis or later reservation is proposed. This series of processing, unlike conventional methods where human receptionists or doctors make individual judgments, achieves consistency, objectivity, and speed in decision-making by statistically and machine-learning-based analysis of vast case data and image features in high-dimensional space. The technical effects of this system include improved accuracy of symptom analysis (reduced misdiagnosis rate), optimization of consultation date and time determination (shortened waiting time), improved efficiency of medical care by switching to online diagnosis (effective use of medical resources), and improved patient experience (reduced burden). Specific application fields include general outpatient care, emergency triage, telemedicine services, and corporate health management systems. In addition, the system contributes to improvements in computer technology itself, such as cooperation between multiple AI models, database integration, and utilization of GPU clusters for real-time processing.

The assistant system according to the embodiment comprises a reception unit, an analysis unit, a determination unit, and a proposal unit. The reception unit inputs symptoms when a patient visits the hospital. The patient only needs to easily input their symptoms. For example, symptoms such as “I have a headache” or “I have a cough” are input. This information is input to the generative AI. The analysis unit analyzes the input symptoms using the generative AI. The generative AI analyzes the patient's symptoms and, in cooperation with the image analysis AI, narrows down the patient's symptoms to a certain extent. For example, if the patient has a headache, the image analysis AI analyzes the patient's complexion and facial expression to determine whether the symptoms are urgent. This enables rapid narrowing down of the patient's symptoms. The determination unit determines the optimal consultation date and time based on the narrowed-down symptoms, existing reservations, and the patient's desired date and time. For example, if the patient wishes to receive a consultation in the morning of the next day, the generative AI checks the existing reservation status and can propose the optimal consultation date and time. This allows the patient to shorten the waiting time. The proposal unit can switch to online diagnosis depending on the diagnosis result by the generative AI. For example, if the symptoms are mild and it is determined that online diagnosis is possible, the generative AI can propose online diagnosis to the patient. As a result, the patient does not need to go to the hospital and can receive consultation efficiently. Thus, the assistant system according to the embodiment can efficiently perform the entire process from symptom input, analysis, determination of consultation date and time, to proposal of online diagnosis. Specifically, the assistant system operates by linking multiple computer modules (reception unit, analysis unit, determination unit, proposal unit) via a network. The reception unit is designed so that patients can input symptoms in natural language or by selection via a user interface such as a touch panel terminal or smartphone application. The input data supports various formats, including text data (e.g., Japanese sentences such as “I have a headache,” “I have a cough,” up to 256 tokens), image data (facial photos or affected area images, RGB image tensor: 224×224×3), and audio data (audio files, 16 kHz, 16 bit, WAV format). These data are preprocessed by a preprocessing unit, which performs noise removal, normalization, tokenization, image resizing, etc., and are then input to the generative AI (for example, a transformer-based large language model or multimodal model). The generative AI vectorizes the input symptom text and image features using multi-layer self-attention mechanisms and convolutional neural networks, and performs symptom classification tasks (e.g., ICD-10 code classification, severity scoring) and urgency estimation tasks (e.g., probability value from 0 to 1, judged as urgent if threshold is 0.7 or higher). The image analysis AI uses CNN architectures such as ResNet or EfficientNet to analyze complexion (e.g., RGB histogram, blood color estimation) and facial expression (e.g., facial muscle feature point extraction, emotion estimation), and feeds back auxiliary features for urgency and severity to the generative AI. The analysis unit integrates these multimodal features and narrows down the symptoms (e.g., outputs the top three candidate diseases in a list, probability distribution for each disease). The determination unit cross-references the hospital's reservation management database (e.g., SQL table, reservation ID, date and time, attending physician, availability, etc.) and the patient's desired date and time (e.g., calendar format, desired date list), and calculates the optimal consultation date and time using a combinatorial optimization algorithm (e.g., constraint satisfaction problem, priority-based scheduling). The output is presented to the patient as a list of candidate dates and times (e.g., 2024/07/01 10:00, 2024/07/01 11:30, etc.) and recommendation scores (0 to 1). Furthermore, the proposal unit, based on the diagnosis result by the generative AI (e.g., labels of mild, moderate, or severe, online diagnosis availability flag), proposes switching to online diagnosis (e.g., generates video call links, automatically generates online diagnosis reservation forms). Examples of AI output include “Input: ‘I have a headache’+facial image→Output: ‘Urgency 0.2, candidate diseases: migraine (0.7), tension-type headache (0.2), cerebral hemorrhage (0.1)’” and “Input: ‘I have a cough’+facial image→Output: ‘Urgency 0.8, candidate diseases: pneumonia (0.6), bronchitis (0.3), common cold (0.1)’”. In subsequent processing, if urgency is high, immediate consultation slots are preferentially allocated, and if low, online diagnosis or later reservation is proposed. This series of processing, unlike conventional methods where human receptionists or doctors make individual judgments, achieves consistency, objectivity, and speed in decision-making by statistically and machine-learning-based analysis of vast case data and image features in high-dimensional space. The technical effects of this system include improved accuracy of symptom analysis (reduced misdiagnosis rate), optimization of consultation date and time determination (shortened waiting time), improved efficiency of medical care by switching to online diagnosis (effective use of medical resources), and improved patient experience (reduced burden). Specific application fields include general outpatient care, emergency triage, telemedicine services, and corporate health management systems. In addition, the system contributes to improvements in computer technology itself, such as cooperation between multiple AI models, database integration, and utilization of GPU clusters for real-time processing.

The reception unit is capable of inputting the symptoms of a patient. The reception unit inputs symptoms when a patient visits the hospital. The patient only needs to easily input their symptoms. For example, symptoms such as “I have a headache” or “I have a cough” are input. This information is input to the generative AI. This enables the patient to easily input symptoms. Specifically, the reception unit is equipped with a user interface such as a touch panel terminal or smartphone application, allowing the patient to input symptoms in various formats, including natural language text (e.g., “I have a headache,” “I have a cough,” up to 256 tokens), image data (facial photos or affected area images, RGB image tensor: 224×224×3), and audio data (16 kHz, 16 bit, WAV format). The reception unit transmits the input data to a preprocessing unit, which automatically performs preprocessing such as noise removal, normalization, tokenization, and image resizing. The preprocessed data is then input to the generative AI (for example, a transformer-based large language model or multimodal model). Examples of input to the AI include “Input: text ‘I have a headache’” and “Input: image (facial photo)”. The AI vectorizes these inputs and performs tasks such as symptom classification and urgency estimation. Examples of AI output include “Urgency 0.2, candidate diseases: migraine (0.7), tension-type headache (0.2), cerebral hemorrhage (0.1)”. By providing such diverse input formats and preprocessing functions, the reception unit enables patients to input symptoms in the most suitable way according to their situation, greatly improving the convenience and accuracy of input. The technical effects include improved efficiency of data acquisition, reduced input errors, and improved patient experience compared to conventional paper-based or single-input methods due to the diverse input support and automatic preprocessing of the reception unit. Application fields include general outpatient reception, emergency reception, telemedicine reception, and corporate health management reception.

The analysis unit is capable of analyzing the symptoms of a patient by cooperation between a generative AI and an image analysis AI. The analysis unit analyzes the symptoms of a patient by cooperation between a generative AI and an image analysis AI. The generative AI analyzes the patient's symptoms and, in cooperation with the image analysis AI, narrows down the patient's symptoms to a certain extent. For example, if the patient has a headache, the image analysis AI analyzes the patient's complexion and facial expression to determine whether the symptoms are urgent. This cooperation between the generative AI and the image analysis AI improves the accuracy of symptom analysis. Specifically, the analysis unit receives multimodal data such as text data (e.g., natural language sentences like “I have a headache”), image data (facial photos or affected area images, RGB image tensor: 224×224×3), and audio data (audio files, 16 kHz, 16 bit, WAV format), and after preprocessing such as noise removal and normalization by a preprocessing unit, inputs them to the generative AI (transformer-based large language model or multimodal model). The generative AI tokenizes the input text, vectorizes it using self-attention mechanisms, and performs symptom classification (e.g., ICD-10 code classification, severity scoring) and urgency estimation (probability value from 0 to 1, judged as urgent if threshold is 0.7 or higher). The image analysis AI uses CNN architectures such as ResNet or EfficientNet to analyze complexion (RGB histogram, blood color estimation) and facial expression (facial muscle feature point extraction, emotion estimation), and feeds back auxiliary features for urgency and severity to the generative AI. The analysis unit integrates these multimodal features and outputs a candidate disease list (e.g., migraine, tension-type headache, cerebral hemorrhage) and probability distribution for each disease. An example of AI input and output is “Input: ‘I have a headache’+facial image→Output: ‘Urgency 0.2, candidate diseases: migraine (0.7), tension-type headache (0.2), cerebral hemorrhage (0.1)’”. In subsequent processing, if urgency is high, immediate consultation slots are preferentially allocated, and if low, online diagnosis or later reservation is proposed. Unlike conventional human interviews or visual judgments, the analysis unit performs statistical and machine-learning-based analysis in high-dimensional space, greatly improving consistency, objectivity, and speed of judgment. The technical effects include improved accuracy of symptom analysis, reduced misdiagnosis rate, and faster diagnosis. Application fields include outpatient care, emergency triage, telemedicine, and health management systems.

The determination unit is capable of determining an appropriate consultation date and time based on existing reservations and the desired date and time of the patient. The determination unit determines an appropriate consultation date and time based on existing reservations and the desired date and time of the patient. Based on the narrowed-down symptoms, the determination unit determines the optimal consultation date and time by considering existing reservations and the desired date and time of the patient. For example, if the patient wishes to receive a consultation in the morning of the next day, the generative AI checks the existing reservation status and can propose the optimal consultation date and time. This enables the determination of the optimal consultation date and time according to the patient's wishes. Specifically, the determination unit receives the candidate disease list and urgency score output from the analysis unit (e.g., urgency 0.8, candidate diseases: pneumonia (0.6), bronchitis (0.3), common cold (0.1)), and cross-references the hospital's reservation management database (SQL table, reservation ID, date and time, attending physician, availability, etc.) and the patient's desired date and time (calendar format, desired date list). The determination unit uses a combinatorial optimization algorithm (e.g., constraint satisfaction problem, priority-based scheduling) to calculate the optimal consultation date and time that simultaneously satisfies multiple constraints such as urgency, patient preferences, and physician availability. Examples of AI input include “candidate disease list+urgency+desired date and time+reservation database,” and examples of output include “candidate date and time list (2024/07/01 10:00, 2024/07/01 11:30, etc.)+recommendation score (0.9, 0.7, etc.).” In subsequent processing, the date and time with the highest recommendation score is presented to the patient, and the date and time selected by the patient is registered in the reservation system. Unlike conventional manual work or simple first-come-first-served allocation by humans, the determination unit simultaneously considers high-dimensional constraints, enabling shortened waiting times and improved efficiency of medical care. The technical effects include optimization of consultation date and time determination, improved patient satisfaction, and effective use of medical resources. Application fields include outpatient reservation management, emergency consultation scheduling, and telemedicine reservation.

The proposal unit is capable of proposing online diagnosis based on the diagnosis result by the generative AI. The proposal unit proposes online diagnosis based on the diagnosis result by the generative AI. Depending on the diagnosis result by the generative AI, the system can switch to online diagnosis. For example, if the symptoms are mild and it is determined that online diagnosis is possible, the generative AI can propose online diagnosis to the patient. By proposing online diagnosis based on the diagnosis result, the efficiency of consultation is improved. Specifically, the proposal unit automatically generates proposals for switching to online diagnosis based on the diagnosis result (e.g., labels of mild, moderate, or severe, urgency score, online diagnosis availability flag) received from the analysis unit and determination unit. Examples of AI input include “diagnosis result label+urgency+patient attribute information,” and examples of output include “online diagnosis recommendation flag (True/False), video call link generation, online diagnosis reservation form auto-generation.” If the proposal unit determines that online diagnosis is appropriate, it explains the advantages of online diagnosis to the patient (e.g., no need to visit the hospital, rapid diagnosis, effective use of medical resources), and, if necessary, automatically generates and presents an online diagnosis reservation form or video call link. In subsequent processing, if the patient selects online diagnosis, the reservation is registered in the system and the physician is notified. Unlike conventional human judgment or manual guidance, the proposal unit performs consistent rule-based processing and automated proposal generation by AI, greatly improving consultation efficiency and patient experience. The technical effects include improved efficiency of medical care, effective use of medical resources, and reduced patient burden. Application fields include telemedicine services, general outpatient care, and corporate health management.

The analysis unit is capable of analyzing the patient's complexion and facial expression by the image analysis AI and determining whether the symptoms are urgent. The analysis unit analyzes the patient's complexion and facial expression by the image analysis AI and determines whether the symptoms are urgent. The image analysis AI analyzes the patient's complexion and facial expression to determine whether the symptoms are urgent. By analyzing the patient's complexion and facial expression, urgent symptoms can be quickly determined. Specifically, the analysis unit uses convolutional neural network (CNN) architectures such as ResNet or EfficientNet as the image analysis AI, and receives input images (facial photos or affected area images, RGB image tensor: 224×224×3). The image analysis AI extracts features through multi-layer convolution and pooling processing, calculates features such as complexion (RGB histogram, blood color estimation) and facial expression (facial muscle feature point extraction, emotion estimation). Examples of AI input include “facial image (RGB tensor),” and examples of output include “complexion score (0.8), facial expression feature (vector), urgency estimation value (0.9).” The analysis unit uses these features to determine with high probability the possibility of urgent symptoms (e.g., cerebral hemorrhage, severe infectious disease), and allocates immediate consultation slots as necessary. Unlike conventional visual judgment or empirical rules by humans, the image analysis AI performs statistical and machine-learning-based analysis in high-dimensional space, greatly improving consistency, objectivity, and speed of judgment. The technical effects include rapid detection of emergency cases, reduced misdiagnosis rate, and improved efficiency of medical care. Application fields include emergency outpatient triage, remote diagnosis, and health management systems.

The reception unit is capable of estimating the patient's emotions and adjusting the symptom input interface based on the estimated emotions of the patient. The reception unit estimates the patient's emotions and adjusts the symptom input interface based on the estimated emotions. For example, if the patient feels anxious, a simple and intuitive interface is provided, minimizing the input steps. If the patient is relaxed, detailed input options are provided, and customizable input methods may also be proposed. Furthermore, if the patient is in a hurry, voice input is prioritized to enable rapid symptom input. By adjusting the interface according to the patient's emotions, symptom input can be performed smoothly. Emotion estimation is realized by using an emotion engine or generative AI, for example, with emotion estimation functions. The generative AI may be a text generative AI (e.g., LLM) or a multimodal generative AI, but is not limited to these examples. Specifically, the reception unit inputs the patient's input text (e.g., “I am anxious,” “I am nervous,” etc.), audio data (tone and speed of voice), and image data (facial expression image) to an emotion estimation AI (e.g., BERT-based emotion classification model or multimodal emotion estimation model). Examples of AI input include “Text: ‘I am anxious’,” “Audio: nervous voice,” “Image: frowning face,” and examples of output include “Emotion label: anxiety, confidence 0.85,” “Emotion label: relaxed, confidence 0.9.” The reception unit automatically switches the layout and input steps of the interface according to the estimated emotion label (e.g., reduces the number of buttons and displays encouraging messages for anxiety, adds detailed input options for relaxation, prioritizes voice input for urgency). Such emotion-adaptive interfaces, unlike conventional uniform screen designs, provide an optimized input experience for each patient. The technical effects include reduced input errors, improved input completion rate, and improved patient satisfaction. Application fields include medical reception terminals, telemedicine apps, and health management apps.

The reception unit is capable of analyzing the patient's past consultation history and proposing an appropriate symptom input method. The reception unit analyzes the patient's past consultation history and proposes an appropriate symptom input method. For example, symptoms frequently input by the patient in the past are automatically displayed as candidates. In addition, input methods (voice, text, etc.) previously used by the patient may be preferentially proposed. Furthermore, symptoms used at specific times based on the patient's past consultation history may be predicted and proposed. By proposing the optimal symptom input method based on past consultation history, input efficiency is improved. Specifically, the reception unit refers to a consultation history database for each patient (e.g., SQL table, structured data such as patient ID, consultation date, symptoms, input method, department, etc.) and analyzes past symptom input history in chronological order. The reception unit extracts frequent symptoms (e.g., text labels such as “headache,” “cough,” top three by frequency), previously selected input methods (e.g., count values for voice input usage, text input usage, image input usage), and symptom occurrence trends by consultation time (e.g., “abdominal pain” tendency in the morning, “insomnia” tendency at night). The reception unit inputs these features to a candidate generation algorithm (e.g., frequency-based candidate list generation, time-conditioned symptom prediction model), and automatically generates a symptom candidate list (e.g., “headache,” “cough,” “abdominal pain,” etc.) and recommended input method (e.g., voice input recommendation flag, text input recommendation flag) according to the patient's current visit time and past trends. Examples of AI input include “Patient ID: 12345, past three symptoms: headache, cough, headache, past input methods: voice, text, voice, visit time: 10 a.m.,” and examples of output include “Candidate symptoms: headache (0.8), cough (0.6), recommended input method: voice (0.7).” The reception unit presents these outputs as candidate buttons or input method switching guidance on the user interface, allowing the patient to select with one touch. In subsequent processing, if the patient selects a symptom from the candidates, the selection is recorded in the consultation database and reflected in candidate generation for future visits. Unlike conventional uniform input screens or non-history-utilizing reception systems, the reception unit analyzes each patient's history data as high-dimensional features and provides an individually optimized input experience, resulting in significant improvement in input efficiency, reduced input errors, and improved patient satisfaction. Application fields include general outpatient reception, chronic disease management clinics, corporate health management systems, and telemedicine reception. In addition, the introduction of time-series analysis of history data and input method recommendation algorithms realizes technical advances in data-driven reception systems beyond mere automation of computer-based reception operations.

The reception unit is capable of filtering input content based on the patient's current health condition and lifestyle habits when inputting symptoms. The reception unit filters input content based on the patient's current health condition and lifestyle habits when inputting symptoms. For example, symptoms related to medications currently being taken by the patient are preferentially input. In addition, symptoms related to the patient's lifestyle habits (smoking, drinking, etc.) may be input. Furthermore, symptoms related to the patient's current health condition (chronic diseases, etc.) may be preferentially input. By filtering input content based on the patient's health condition and lifestyle habits, appropriate symptom input can be performed. Specifically, the reception unit refers to a health condition database for each patient (e.g., structured data such as chronic disease flags, medication history, medical history, allergy information) and a lifestyle habits database (e.g., smoking status, drinking frequency, exercise habits, dietary patterns). The reception unit inputs these data to a symptom candidate generation AI (e.g., rule-based+machine learning hybrid model), and dynamically generates a symptom candidate list according to the patient's attributes. Examples of AI input include “Medication: antihypertensive, chronic disease: hypertension, lifestyle: smoker, non-drinker,” and examples of output include “Priority symptom candidates: dizziness (0.7), palpitations (0.6), cough (0.4).” The reception unit highlights these candidates on the interface to make them easy for the patient to select. Furthermore, as a filtering process for input content, the system implements an algorithm to exclude symptoms unrelated to the patient's health condition (e.g., symptoms specific to children) from the candidate list. In subsequent processing, symptoms selected by the patient are recorded in the consultation database and used for future candidate generation and health management proposals. Unlike conventional uniform symptom input screens or non-attribute-considering reception systems, the reception unit analyzes each patient's health condition and lifestyle habits as high-dimensional features and provides an individually optimized symptom input experience, resulting in improved input accuracy, reduced input errors, and improved patient satisfaction. Application fields include chronic disease outpatient care, lifestyle disease clinics, corporate health management reception, and telemedicine reception. In addition, the integration of health condition and lifestyle habit data analysis and symptom candidate filtering algorithms realizes technical advances in data-driven reception systems beyond mere automation of computer-based reception operations.

The reception unit is capable of estimating the patient's emotions and determining the priority order of symptoms to be input based on the estimated emotions of the patient. The reception unit estimates the patient's emotions and determines the priority order of symptoms to be input based on the estimated emotions. For example, if the patient feels anxious, symptoms with high urgency are preferentially input. If the patient is relaxed, options for inputting detailed symptoms may be provided. Furthermore, if the patient is in a hurry, only the main symptoms may be preferentially input. By determining the priority order of symptoms according to the patient's emotions, appropriate symptom input can be performed. Emotion estimation is realized by using an emotion engine or generative AI, for example, with emotion estimation functions. The generative AI may be a text generative AI (e.g., LLM) or a multimodal generative AI, but is not limited to these examples. Specifically, the reception unit inputs the patient's input text (e.g., “I am anxious,” “I am in a hurry,” etc.), audio data (tone and speed of voice, 16 kHz, 16 bit, WAV format), and image data (facial expression image, RGB image tensor: 224×224×3) to an emotion estimation AI (e.g., BERT-based emotion classification model, multimodal emotion estimation model). Examples of AI input include “Text: ‘I am anxious’,” “Audio: nervous voice,” “Image: frowning face,” and examples of output include “Emotion label: anxiety, confidence 0.85,” “Emotion label: relaxed, confidence 0.9.” The reception unit automatically switches the order and content of the symptom input candidate list according to the estimated emotion label (e.g., displays urgent symptoms at the top for anxiety, adds detailed symptom input options for relaxation, displays only main symptoms for urgency). In subsequent processing, symptoms selected by the patient are recorded in the consultation database and used for future candidate generation and optimization of the emotion-adaptive interface. Unlike conventional uniform symptom input screens or non-emotion-considering reception systems, the reception unit analyzes the patient's emotional state as high-dimensional features and provides an individually optimized symptom input experience, resulting in improved input efficiency, reduced input errors, and improved patient satisfaction. Application fields include medical reception terminals, telemedicine apps, and health management apps. In addition, the cooperation between emotion estimation AI and symptom input candidate generation algorithms realizes technical advances in emotion-adaptive reception systems beyond mere automation of computer-based reception operations.

The reception unit is capable of prioritizing input of highly relevant symptoms based on the patient's geographic location information when inputting symptoms. The reception unit prioritizes input of highly relevant symptoms based on the patient's geographic location information when inputting symptoms. For example, if the patient lives in a specific region, symptoms related to diseases prevalent in that region are preferentially input. In addition, if the patient is traveling, symptoms related to health risks at the travel destination may be preferentially input. Furthermore, if the patient works in a specific environment (factory, farm, etc.), symptoms related to that environment may be preferentially input. By considering the patient's geographic location information when inputting symptoms, appropriate symptom input can be performed. Specifically, the reception unit integrally refers to the patient's current location information (e.g., GPS coordinates, address data), workplace environment information (e.g., environment labels such as factory, farm, office), and regional epidemic disease database (e.g., regional infectious disease epidemic status, environmental risk information). The reception unit inputs these data to a symptom candidate generation AI (e.g., geographic information-linked symptom prediction model), and dynamically generates a symptom candidate list according to the patient's current location and environment. Examples of AI input include “Location: Shinjuku, Tokyo, environment: office, season: summer” or “Location: rural Hokkaido, environment: farm, season: autumn,” and examples of output include “Priority symptom candidates: heatstroke (0.8), influenza (0.6), pesticide poisoning (0.5).” The reception unit highlights these candidates on the interface to make them easy for the patient to select. In subsequent processing, symptoms selected by the patient are recorded in the consultation database and used for statistical analysis of regional epidemic status and public health measures. Unlike conventional uniform symptom input screens or non-geographic-information-considering reception systems, the reception unit analyzes the patient's geographic location information and environment information as high-dimensional features and provides an individually optimized symptom input experience, resulting in improved input accuracy, early detection of region-specific diseases, and improved patient satisfaction. Application fields include outpatient reception during infectious disease epidemics, traveler health management systems, and occupational health reception. In addition, the introduction of geographic information-linked symptom candidate generation algorithms realizes technical advances in region-adaptive reception systems beyond mere automation of computer-based reception operations.

The reception unit is capable of analyzing the patient's social media activity and inputting relevant symptoms when inputting symptoms. The reception unit analyzes the patient's social media activity and inputs relevant symptoms when inputting symptoms. For example, symptoms related to health information shared by the patient on social media are input. In addition, symptoms related to information obtained from health-related accounts followed by the patient on social media may be input. Furthermore, symptoms related to information obtained from health-related groups the patient participates in on social media may be input. By analyzing the patient's social media activity, relevant symptoms can be appropriately input. Specifically, the reception unit, with the patient's consent, obtains the patient's posting history (e.g., health-related post text, images, videos), followed account list (e.g., medical institutions, health information providers), and group participation information (e.g., diabetes patient association, smoking cessation support group, etc.) via social media APIs. The reception unit inputs these data to a natural language processing AI (e.g., BERT-based health-related text classification model, multimodal post analysis model), and extracts health status and symptoms of interest from post content and follow trends. Examples of AI input include “Post text: ‘I've had a persistent cough lately,’ ‘My headache is severe,’” “Followed account: asthma patient association,” “Group participation: hay fever countermeasures,” and examples of output include “Related symptom candidates: cough (0.9), headache (0.8), hay fever (0.7).” The reception unit highlights these candidates on the interface to make them easy for the patient to select. In subsequent processing, symptoms selected by the patient are recorded in the consultation database and used for health management proposals and preventive education. Unlike conventional uniform symptom input screens or non-social-media-utilizing reception systems, the reception unit analyzes the patient's online activity data as high-dimensional features and provides an individually optimized symptom input experience, resulting in improved input accuracy, increased health awareness, and improved patient satisfaction. Application fields include health management apps for young people, chronic disease patient support systems, and telemedicine reception. In addition, the introduction of social media-linked symptom candidate generation algorithms realizes technical advances in online-behavior-adaptive reception systems beyond mere automation of computer-based reception operations.

The analysis unit is capable of estimating the patient's emotions and adjusting the accuracy of symptom analysis based on the estimated emotions of the patient. The analysis unit estimates the patient's emotions and adjusts the accuracy of symptom analysis based on the estimated emotions. For example, if the patient feels anxious, symptoms with high urgency are preferentially analyzed. If the patient is relaxed, detailed symptom analysis may be performed. Furthermore, if the patient is in a hurry, only the main symptoms may be preferentially analyzed. By adjusting the accuracy of symptom analysis according to the patient's emotions, appropriate analysis can be performed. Emotion estimation is realized by using an emotion engine or generative AI, for example, with emotion estimation functions. The generative AI may be a text generative AI (e.g., LLM) or a multimodal generative AI, but is not limited to these examples. Specifically, the analysis unit inputs the patient's input text (e.g., “I am anxious,” “I am relaxed,” “I am in a hurry,” etc., up to 256 tokens), audio data (16 kHz, 16 bit, WAV format, tone and speed of voice), and image data (facial expression image, RGB image tensor: 224×224×3) to an emotion estimation AI (e.g., BERT-based emotion classification model or multimodal emotion estimation model). Examples of AI input include “Text: ‘I am anxious’,” “Audio: nervous voice,” “Image: frowning face,” and the AI vectorizes these inputs through preprocessing such as tokenization, spectral analysis, and image feature extraction, and outputs emotion labels (e.g., anxiety, relaxation, urgency) and confidence scores (0.0 to 1.0). Examples of output include “Emotion label: anxiety, confidence 0.85,” “Emotion label: relaxed, confidence 0.9.” The analysis unit dynamically switches the parameters and inference flow of the symptom analysis AI (e.g., transformer-based large language model or multimodal model) according to the estimated emotion label. For example, in the case of anxiety, the weight of the urgency estimation task is increased, and symptoms with high urgency (e.g., cerebral hemorrhage, severe infectious disease) are preferentially analyzed, while detailed symptom classification is omitted. In the case of relaxation, the top five candidate diseases in the list are analyzed in detail, and severity scores and auxiliary risk factors are also output. In the case of urgency, only the main symptoms are quickly analyzed, and a simple candidate disease list (top one or two) and urgency score are output. Examples of AI output include “Input: ‘I am anxious’+headache→Output: urgency 0.9, candidate diseases: cerebral hemorrhage (0.7), migraine (0.2)” and “Input: ‘I am relaxed’+cough→Output: urgency 0.3, candidate diseases: common cold (0.6), bronchitis (0.3), pneumonia (0.1).” In subsequent processing, if urgency is high, immediate consultation slots are preferentially allocated, and if low, online diagnosis or later reservation is proposed. Unlike conventional uniform analysis flows or subjective human judgment, the analysis unit dynamically optimizes the behavior of the analysis AI by analyzing the patient's emotional state as high-dimensional features, resulting in improved analysis accuracy, reduced misdiagnosis rate, and improved patient experience. Application fields include emergency outpatient triage, remote medical diagnosis, and health management systems. In addition, the introduction of cooperation between emotion estimation AI and symptom analysis AI and dynamic parameter optimization algorithms realizes technical advances in emotion-adaptive analysis systems beyond mere automation of computer-based medical analysis operations.

The analysis unit is capable of optimizing the analysis algorithm by referring to the patient's past consultation data when analyzing symptoms. The analysis unit optimizes the analysis algorithm by referring to the patient's past consultation data when analyzing symptoms. For example, the accuracy of symptom analysis is improved based on the patient's past consultation data. In addition, specific patterns may be extracted from the patient's past consultation data to optimize the analysis algorithm. Furthermore, the priority order of symptom analysis may be determined by referring to the patient's past consultation data. By referring to the patient's past consultation data, the analysis algorithm can be optimized. Specifically, the analysis unit refers to a consultation history database for each patient (e.g., SQL table, structured data such as patient ID, consultation date, symptoms, diagnosis result, treatment content, test values) and extracts past symptom, diagnosis, and treatment patterns in chronological order. The analysis unit extracts features such as frequent symptoms (e.g., text labels such as “headache,” “cough,” top three by frequency), past diagnosis results (e.g., ICD-10 code, severity score), and treatment responsiveness (e.g., improvement score after treatment), and inputs them to the symptom analysis AI (e.g., transformer-based large language model or multimodal model). Examples of AI input include “Patient ID: 12345, past three symptoms: headache, cough, headache, past diagnoses: migraine, common cold, migraine, treatment response: good, normal, good,” and the AI vectorizes these history features and dynamically optimizes parameters for the current symptom analysis task (e.g., weights for symptom classification, threshold for urgency estimation, order of candidate disease list). Examples of output include “Candidate diseases: migraine (0.8), tension-type headache (0.15), cerebral hemorrhage (0.05)” and “Analysis priority: headache (0.9), cough (0.6).” Furthermore, time-series patterns (e.g., seasonal onset trends, recurrence cycles) may be extracted from past consultation data, and algorithms for automatically adjusting the priority and scope of symptom analysis are also implemented. In subsequent processing, the analysis results are recorded in the consultation database and reflected in optimization of the analysis algorithm for future cases. Unlike conventional uniform analysis flows or non-history-utilizing analysis systems, the analysis unit dynamically optimizes the behavior of the analysis AI by analyzing each patient's history data as high-dimensional features, resulting in improved analysis accuracy, reduced misdiagnosis rate, and individual optimization for each patient. Application fields include chronic disease management clinics, outpatient care for recurrent diseases, and remote medical diagnosis. In addition, the introduction of time-series analysis of history data and parameter optimization algorithms realizes technical advances in history-adaptive analysis systems beyond mere automation of computer-based medical analysis operations.

The analysis unit is capable of performing analysis based on the patient's living environment and occupational information when analyzing symptoms. The analysis unit performs analysis based on the patient's living environment and occupational information when analyzing symptoms. For example, symptom analysis is performed by considering the patient's living environment (urban, rural, etc.). In addition, symptoms related to the patient's occupational information (factory worker, office worker, etc.) may be analyzed. Furthermore, symptom analysis may be performed by considering specific risk factors based on the patient's living environment and occupational information. By considering the patient's living environment and occupational information, appropriate symptom analysis can be performed. Specifically, the analysis unit refers to a living environment database (e.g., residence label, urban/rural classification, housing environment, number of cohabitants) and an occupational information database (e.g., occupation code, work style, work environment label, working hours) for each patient, and inputs these data to a symptom analysis AI (e.g., geography/occupation-linked multimodal model). Examples of AI input include “Residence: urban, occupation: factory worker, working hours: night shift,” “Residence: rural, occupation: agricultural worker, working hours: daytime,” and the AI vectorizes these environment and occupation features and reflects them in symptom analysis tasks (e.g., candidate disease list generation, risk factor estimation, severity scoring). Examples of output include “Candidate diseases: heatstroke (0.7), asthma (0.5), pesticide poisoning (0.3)” and “Risk factors: organic solvent exposure (0.6), night shift stress (0.4).” Furthermore, algorithms for symptom analysis that consider specific environmental risks (e.g., air pollution, pesticide exposure, noise, stress) based on living environment and occupational information (e.g., environmental risk weighting, occupation-based symptom priority adjustment) are also implemented. In subsequent processing, the analysis results are recorded in the consultation database and used for environment/occupation risk management and health guidance proposals. Unlike conventional uniform analysis flows or non-environment/occupation-considering analysis systems, the analysis unit dynamically optimizes the behavior of the analysis AI by analyzing each patient's living environment and occupational information as high-dimensional features, resulting in improved analysis accuracy, early detection of risk factors, and individual optimization for each patient. Application fields include occupational health diagnosis, environmental risk outpatient care, and remote medical diagnosis. In addition, the introduction of environment/occupation-linked analysis algorithms realizes technical advances in environment-adaptive analysis systems beyond mere automation of computer-based medical analysis operations.

The analysis unit is capable of estimating the patient's emotions and adjusting the display method of analysis results based on the estimated emotions of the patient. The analysis unit estimates the patient's emotions and adjusts the display method of analysis results based on the estimated emotions. For example, if the patient feels anxious, a simple and highly visible display method is provided. If the patient is relaxed, a display method including detailed information may be provided. Furthermore, if the patient is in a hurry, a display method focusing on key points may be provided. By adjusting the display method of analysis results according to the patient's emotions, appropriate information provision can be performed. Emotion estimation is realized by using an emotion engine or generative AI, for example, with emotion estimation functions. The generative AI may be a text generative AI (e.g., LLM) or a multimodal generative AI, but is not limited to these examples. Specifically, the analysis unit inputs the patient's input text (e.g., “I am anxious,” “I am relaxed,” “I am in a hurry,” etc.), audio data (16 kHz, 16 bit, WAV format), and image data (facial expression image, RGB image tensor: 224×224×3) to an emotion estimation AI (e.g., BERT-based emotion classification model, multimodal emotion estimation model), and outputs emotion labels (e.g., anxiety, relaxation, urgency) and confidence scores. Examples of AI input include “Text: ‘I am anxious’,” “Audio: nervous voice,” “Image: frowning face,” and examples of output include “Emotion label: anxiety, confidence 0.85,” “Emotion label: relaxed, confidence 0.9.” The analysis unit dynamically switches the layout, amount of information, and highlighted items of the analysis result display module according to the estimated emotion label. For example, in the case of anxiety, only the main diagnosis result and urgency are displayed simply in large font, and detailed information is hidden. In the case of relaxation, detailed information such as candidate disease list, severity score, risk factors, and recommended treatment is provided in tab format or expandable display. In the case of urgency, only the key points (e.g., most suspected disease name and urgency score) are displayed concisely, and operation steps are minimized. In subsequent processing, if the patient requests detailed information, additional information is displayed stepwise, and the display history is recorded in the consultation database. Unlike conventional uniform display screens or non-emotion-considering information provision, the analysis unit dynamically optimizes the behavior of the analysis result display module by analyzing the patient's emotional state as high-dimensional features, resulting in improved efficiency of information transmission, improved patient satisfaction, and reduced misunderstanding and anxiety. Application fields include medical reception terminals, telemedicine apps, and health management apps. In addition, the cooperation between emotion estimation AI and display control algorithms realizes technical advances in emotion-adaptive information provision systems beyond mere automation of computer-based medical information provision operations.

The analysis unit is capable of performing analysis based on the patient's geographic distribution when analyzing symptoms. The analysis unit performs analysis based on the patient's geographic distribution when analyzing symptoms. For example, symptom analysis is performed by considering the health risks of the region where the patient lives. In addition, symptoms related to diseases prevalent in specific regions may be analyzed based on the patient's geographic distribution. Furthermore, symptom analysis may be performed by considering specific environmental factors (air pollution, water quality, etc.) based on the patient's geographic distribution. By considering the patient's geographic distribution, appropriate symptom analysis can be performed. Specifically, the analysis unit integrally refers to the patient's current location information (e.g., GPS coordinates, address data), residential region label (e.g., prefecture, city/ward/town/village), and regional epidemic disease database (e.g., regional infectious disease epidemic status, environmental risk information), and inputs these data to a symptom analysis AI (e.g., geographic information-linked multimodal model). Examples of AI input include “Location: Shinjuku, Tokyo, season: summer,” “Location: rural Hokkaido, season: autumn,” and the AI vectorizes these geographic features and reflects them in symptom analysis tasks (e.g., candidate disease list generation, regional risk factor estimation, severity scoring). Examples of output include “Candidate diseases: heatstroke (0.8), influenza (0.6), pesticide poisoning (0.5)” and “Environmental risk: air pollution (0.7), water quality deterioration (0.4).” Furthermore, algorithms for symptom analysis that consider specific regional risks (e.g., infectious disease epidemics, pollen dispersion, PM2.5 concentration increase) based on geographic distribution (e.g., regional risk weighting, seasonal disease priority adjustment) are also implemented. In subsequent processing, the analysis results are recorded in the consultation database and used for statistical analysis of regional epidemic status and public health measures. Unlike conventional uniform analysis flows or non-geographic-information-considering analysis systems, the analysis unit dynamically optimizes the behavior of the analysis AI by analyzing the patient's geographic distribution as high-dimensional features, resulting in improved analysis accuracy, early detection of region-specific diseases, and individual optimization for each patient. Application fields include outpatient diagnosis during infectious disease epidemics, traveler health management systems, and occupational health diagnosis. In addition, the introduction of geographic information-linked analysis algorithms realizes technical advances in region-adaptive analysis systems beyond mere automation of computer-based medical analysis operations.

The analysis unit is capable of improving the accuracy of analysis by referring to related literature of the patient when analyzing symptoms. The analysis unit improves the accuracy of analysis by referring to related literature of the patient when analyzing symptoms. For example, the accuracy of analysis is improved by referring to the latest medical literature related to the patient's symptoms. In addition, the analysis algorithm may be optimized based on past research data related to the patient's symptoms. Furthermore, symptom analysis may be performed by considering specific risk factors by referring to literature related to the patient's symptoms. By referring to related literature of the patient, the accuracy of analysis is improved. Specifically, the analysis unit refers to medical literature databases (e.g., PubMed, domestic and international academic papers, case report databases) and research databases (e.g., clinical trial data, meta-analysis results) via API, and inputs literature information related to the patient's symptoms and diagnosis candidates (e.g., title, abstract, keywords, incidence, treatment outcomes) to a natural language processing AI (e.g., BERT-based medical literature search model, knowledge graph-linked model). Examples of AI input include “Symptoms: headache, fever, literature search keywords: cerebral hemorrhage, migraine,” “Symptoms: cough, dyspnea, literature search keywords: pneumonia, bronchitis,” and the AI tokenizes these inputs and calculates relevance scores and evidence levels. Examples of output include “Related literature title: ‘Early Symptoms and Diagnostic Accuracy of Cerebral Hemorrhage,’ relevance 0.9,” “Recommended diagnostic algorithm: ICD-10 code classification+severity scoring.” The analysis unit reflects these literature information in parameter optimization of the symptom analysis AI and candidate disease list generation, realizing analysis based on the latest medical knowledge and evidence. Furthermore, specific risk factors (e.g., new infectious disease outbreaks, emergence of drug-resistant bacteria) are extracted from literature and incorporated into the analysis algorithm to strengthen responsiveness to unknown risks. In subsequent processing, the analysis results are recorded in the consultation database and used for evidence presentation to physicians and automatic generation of patient explanation materials. Unlike conventional analysis based on empirical rules or limited knowledge bases, the analysis unit dynamically optimizes the behavior of the analysis AI by analyzing vast medical literature and research data as high-dimensional features, resulting in improved analysis accuracy, reduced misdiagnosis rate, and rapid reflection of the latest medical knowledge. Application fields include specialized outpatient diagnosis support, rare disease diagnosis, and remote medical diagnosis. In addition, the introduction of literature-linked analysis algorithms and knowledge graph cooperation realizes technical advances in knowledge-adaptive analysis systems beyond mere automation of computer-based medical analysis operations.

The determination unit can estimate the emotions of the patient and adjust the method for determining the consultation date and time based on the estimated emotions of the patient. The determination unit estimates the emotions of the patient and adjusts the method for determining the consultation date and time based on the estimated emotions. For example, if the patient feels anxious, the determination unit preferentially proposes an earlier consultation date and time. If the patient is relaxed, the determination unit can propose a consultation date and time that matches the patient's preferences. Furthermore, if the patient is in a hurry, the determination unit can propose the earliest possible consultation date and time. By adjusting the method for determining the consultation date and time according to the patient's emotions, an appropriate consultation date and time can be proposed. Emotion estimation is realized, for example, by using an emotion estimation function such as an emotion engine or generative AI. The generative AI may be a text generation AI (e.g., LLM) or a multimodal generative AI, but is not limited to these examples. Specifically, the determination unit inputs the patient's text input (e.g., natural language sentences such as “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). Examples of input to the AI include “Text: ‘I am anxious’”, “Voice: tense voice”, “Image: frowning face”; the AI vectorizes these inputs through preprocessing such as tokenization, spectral analysis, and image feature extraction, and outputs emotion labels (e.g., anxious, relaxed, in a hurry) and confidence scores (0.0-1.0). Examples of output include “Emotion label: anxious, confidence 0.85”, “Emotion label: relaxed, confidence 0.9”. The determination unit dynamically switches the parameters and branching flow of the consultation date and time determination algorithm (e.g., priority scheduling, constraint satisfaction problem solver) according to the estimated emotion label. For example, in the case of anxiety, the weights for urgency and early consultation preference are increased, and the shortest available slots are preferentially extracted from the reservation management database (SQL table, reservation ID, date and time, attending physician, availability, etc.), generating a candidate date and time list (e.g., 2024/07/01 09:00, 2024/07/01 10:00) and recommendation scores (0.95, 0.85). In the case of relaxation, the patient's desired date and time (calendar format, desired date list) is emphasized, and the date and time closest to the preference is proposed. In the case of being in a hurry, immediate consultation slots or waiting list slots are automatically searched, and only the earliest date and time is presented. Examples of AI output include “Input: emotion label: anxious+desired date and time: morning of 7/1→Output: candidate date and time: 7/1 09:00 (0.95), 7/1 10:00 (0.85)”, “Input: emotion label: in a hurry→Output: candidate date and time: today 15:00 (0.99)”. In subsequent processing, when the patient selects a proposed date and time, that date and time is registered in the reservation system and notified to the physician. Unlike conventional uniform date and time determination or subjective human judgment, the determination unit analyzes the patient's emotional state as high-dimensional features in cooperation with the analysis AI and dynamically optimizes the behavior of the consultation date and time determination algorithm, thereby achieving technical effects such as improved patient satisfaction, reduced waiting times, and increased efficiency of medical care. Applicable fields include outpatient reservation management, emergency consultation scheduling, and remote consultation reservations. Furthermore, by introducing coordinated control between the emotion estimation AI and the consultation date and time determination algorithm and dynamic optimization of parameters, technical advances in emotion-adaptive reservation systems that go beyond mere automation of medical reservation operations by computers are realized.

The determination unit can refer to the patient's past reservation history when determining the consultation date and time and select the optimal date and time. The determination unit refers to the patient's past reservation history when determining the consultation date and time and selects the optimal date and time. For example, the determination unit proposes the optimal consultation date and time based on the patient's past reservation history. If reservations are concentrated in a specific time slot according to the patient's past reservation history, the determination unit can propose dates and times that avoid that time slot. Furthermore, the determination unit can propose a consultation date and time that matches the patient's preferences by referring to the patient's past reservation history. By referring to the patient's past reservation history, the optimal consultation date and time can be selected. Specifically, the determination unit refers to a reservation history database for each patient (e.g., SQL table, structured data such as patient ID, reservation date, reservation time, department, attending physician, visit status, etc.) and extracts past reservation patterns and visit tendencies in chronological order. The determination unit extracts features such as frequently reserved time slots (e.g., time slot labels such as morning, evening, top 3 by frequency), past reservation cancellation rate, visit rate, and patient preference tendencies (e.g., weekday/holiday, morning/afternoon) from the history data and inputs them into a consultation date and time determination AI (e.g., history-adaptive scheduling model). Examples of input to the AI include “Patient ID: 12345, last 3 reservations: 7/1 10:00, 7/8 10:00, 7/15 11:00, visit status: ◯, ◯, ×”; the AI vectorizes these history features and dynamically optimizes parameters for the current consultation date and time determination task (e.g., weighting of candidate dates and times, congestion avoidance flag, priority of desired date and time). Examples of output include “Candidate dates and times: 7/22 10:00 (0.9), 7/22 11:00 (0.8), congestion avoidance recommendation: 11:00”. Furthermore, the determination unit can automatically detect congested time slots (e.g., Monday mornings are always crowded) from past reservation history and apply a congestion avoidance algorithm to exclude those slots from the candidate date and time list. In subsequent processing, the date and time selected by the patient is registered in the reservation system and reflected in candidate generation and congestion prediction for future appointments. Unlike conventional uniform date and time proposals or reservation systems that do not utilize history, the determination unit analyzes each patient's history data as high-dimensional features and realizes individually optimized consultation date and time proposals, thereby achieving technical effects such as reduced waiting times, alleviated congestion, and improved patient satisfaction. Applicable fields include outpatient reservation management, chronic disease management clinics, and remote consultation reservations. Furthermore, by introducing time-series analysis of history data and congestion avoidance algorithms, technical advances in history-adaptive reservation systems that go beyond mere automation of medical reservation operations by computers are realized.

The determination unit can determine the consultation date and time based on the patient's current living situation and schedule when determining the consultation date and time. The determination unit determines the consultation date and time based on the patient's current living situation and schedule when determining the consultation date and time. For example, the determination unit proposes the optimal consultation date and time by considering the patient's current living situation (work, family, etc.). The determination unit can also refer to the patient's schedule and propose a consultation date and time during available time slots. Furthermore, the determination unit can determine the priority order of consultation dates and times based on the patient's living situation and schedule. By considering the patient's living situation and schedule, the optimal consultation date and time can be determined. Specifically, the determination unit refers to a living situation database for each patient (e.g., occupation, working hours, family structure, childcare/caregiving status, etc., structured data) and a schedule database (e.g., calendar integration API, Google Calendar ICS data, schedule list). The determination unit inputs these data into a schedule-adaptive consultation date and time determination AI (e.g., constraint satisfaction problem solver+machine learning model) and automatically extracts the patient's available time slots and living constraints (e.g., working on weekday mornings, childcare in the afternoon, etc.). Examples of input to the AI include “Working hours: 9:00-17:00, calendar availability: 7/22 17:30-18:30, family structure: two children”; the AI vectorizes these features and outputs a list of candidate consultation dates and times (e.g., 7/22 17:30, 7/23 18:00) and recommendation scores (0.9, 0.8). Furthermore, based on living situation and schedule, the determination unit applies a priority scheduling algorithm and automatically selects the date and time that minimizes the patient's burden. In subsequent processing, the date and time selected by the patient is registered in the reservation system, and any changes in living situation or schedule are reflected in candidate generation for future appointments. Unlike conventional uniform date and time proposals or reservation systems that do not consider living situation, the determination unit analyzes each patient's living situation and schedule as high-dimensional features and realizes individually optimized consultation date and time proposals, thereby achieving technical effects such as improved patient satisfaction, reduced reservation cancellation rates, and increased efficiency of medical care. Applicable fields include outpatient reservation management, work-life balance-oriented clinics, and remote consultation reservations. Furthermore, by introducing schedule integration algorithms and living situation-adaptive scheduling, technical advances in living situation-adaptive reservation systems that go beyond mere automation of medical reservation operations by computers are realized.

The determination unit can estimate the emotions of the patient and determine the priority order of consultation dates and times based on the estimated emotions of the patient. The determination unit estimates the emotions of the patient and determines the priority order of consultation dates and times based on the estimated emotions. For example, if the patient feels anxious, the determination unit preferentially proposes an earlier consultation date and time. If the patient is relaxed, the determination unit can propose a consultation date and time that matches the patient's preferences. Furthermore, if the patient is in a hurry, the determination unit can propose the earliest possible consultation date and time. By determining the priority order of consultation dates and times according to the patient's emotions, an appropriate consultation date and time can be proposed. Emotion estimation is realized, for example, by using an emotion estimation function such as an emotion engine or generative AI. The generative AI may be a text generation AI (e.g., LLM) or a multimodal generative AI, but is not limited to these examples. Specifically, the determination unit inputs the patient's text input (e.g., natural language sentences such as “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). Examples of input to the AI include “Text: ‘I am anxious’”, “Voice: tense voice”, “Image: frowning face”; the AI vectorizes these inputs through preprocessing such as tokenization, spectral analysis, and image feature extraction, and outputs emotion labels (e.g., anxious, relaxed, in a hurry) and confidence scores (0.0-1.0). Examples of output include “Emotion label: anxious, confidence 0.85”, “Emotion label: relaxed, confidence 0.9”. The determination unit dynamically adjusts the order and recommendation scores of the candidate consultation date and time list according to the estimated emotion label. For example, in the case of anxiety, the weights for urgency and early consultation preference are increased, and the candidate date and time list (e.g., 2024/07/01 09:00, 2024/07/01 10:00) is displayed in order of earliest dates and times. In the case of relaxation, the patient's desired date and time is emphasized, and the date and time closest to the preference is displayed at the top. In the case of being in a hurry, immediate consultation slots or waiting list slots are displayed at the top. Examples of AI output include “Input: emotion label: anxious+desired date and time: morning of 7/1→Output: candidate date and time: 7/1 09:00 (0.95), 7/1 10:00 (0.85)”, “Input: emotion label: in a hurry→Output: candidate date and time: today 15:00 (0.99)”. In subsequent processing, the date and time selected by the patient is registered in the reservation system and reflected in candidate generation and optimization of the emotion-adaptive interface for future appointments. Unlike conventional uniform candidate display or reservation systems that do not consider emotions, the determination unit analyzes the patient's emotional state as high-dimensional features and realizes individually optimized consultation date and time proposals, thereby achieving technical effects such as improved patient satisfaction, reduced waiting times, and increased efficiency of medical care. Applicable fields include outpatient reservation management, emergency consultation scheduling, and remote consultation reservations. Furthermore, by introducing coordinated control between the emotion estimation AI and candidate generation algorithm and dynamic optimization of parameters, technical advances in emotion-adaptive reservation systems that go beyond mere automation of medical reservation operations by computers are realized.

The determination unit can select the optimal consultation date and time by considering the patient's geographic location information when determining the consultation date and time. The determination unit selects the optimal consultation date and time by considering the patient's geographic location information when determining the consultation date and time. For example, the determination unit proposes the optimal consultation date and time by considering the traffic conditions in the patient's residential area. The determination unit can also propose a consultation date and time during time slots that are convenient for commuting based on the patient's geographic location information. Furthermore, the determination unit can determine the priority order of consultation dates and times by considering the patient's geographic location information. By considering the patient's geographic location information, the optimal consultation date and time can be selected. Specifically, the determination unit integrally refers to the patient's current location information (e.g., GPS coordinates, address data), residential area label (e.g., prefecture, city/ward/town/village), and regional traffic information database (e.g., public transportation timetables, road congestion status, weather information). The determination unit inputs this information into a geographic information-linked consultation date and time determination AI (e.g., geographic information system-linked scheduling model) and automatically extracts the patient's available commuting time slots and traffic risks (e.g., congestion, service suspension, bad weather, etc.). Examples of input to the AI include “Location: Shinjuku, Tokyo, transportation: train, weather: rain”, “Location: rural Hokkaido, transportation: private car, weather: clear”; the AI vectorizes these geographic features and outputs a list of candidate consultation dates and times (e.g., 7/22 10:00, 7/22 13:00) and recommendation scores (0.9, 0.8). Furthermore, based on geographic location information, the determination unit preferentially proposes time slots that are convenient for commuting (e.g., avoiding rush hours, public transportation operating hours) and reduces the patient's burden. In subsequent processing, the date and time selected by the patient is registered in the reservation system, and any changes in regional traffic conditions are reflected in candidate generation for future appointments. Unlike conventional uniform date and time proposals or reservation systems that do not consider geographic information, the determination unit analyzes the patient's geographic location information as high-dimensional features and realizes individually optimized consultation date and time proposals, thereby achieving technical effects such as reduced commuting burden, reduced reservation cancellation rates, and improved patient satisfaction. Applicable fields include outpatient reservation management in wide-area medical zones, scheduling for local medical institutions, and remote consultation reservations. Furthermore, by introducing geographic information-linked scheduling algorithms, technical advances in geographic-adaptive reservation systems that go beyond mere automation of medical reservation operations by computers are realized.

The determination unit can analyze the patient's social media activity when determining the consultation date and time and propose the optimal date and time. The determination unit analyzes the patient's social media activity when determining the consultation date and time and proposes the optimal date and time. For example, the determination unit confirms that the patient is active during specific time slots based on social media activity and proposes a consultation date and time during those time slots. The determination unit can also propose a consultation date and time that avoids busy time slots based on the patient's social media activity. Furthermore, the determination unit can analyze the patient's social media activity and propose the optimal consultation date and time. By analyzing the patient's social media activity, the optimal consultation date and time can be proposed. Specifically, with the patient's consent, the determination unit obtains the patient's posting history (e.g., posting time, content, images), active time slots (e.g., weekday evenings, weekend mornings), and event participation schedule (e.g., calendar-linked events) via social media APIs. The determination unit inputs these data into a natural language processing AI (e.g., BERT-based posting analysis model, time-series activity prediction model) and automatically extracts the patient's life rhythm and busy time slots. Examples of input to the AI include “Posting history: many posts between 21:00-23:00 on weekdays, event participation on weekend mornings”; the AI vectorizes these features and outputs a list of candidate consultation dates and times (e.g., weekday 18:00, weekend 14:00) and recommendation scores (0.85, 0.8). Furthermore, based on social media activity, the determination unit automatically avoids busy time slots (e.g., during event participation, during meetings) and preferentially proposes available time slots. In subsequent processing, the date and time selected by the patient is registered in the reservation system and reflected in candidate generation and activity analysis for future appointments. Unlike conventional uniform date and time proposals or reservation systems that do not utilize social media, the determination unit analyzes the patient's online activity data as high-dimensional features and realizes individually optimized consultation date and time proposals, thereby achieving technical effects such as improved patient satisfaction, reduced reservation cancellation rates, and increased efficiency of medical care. Applicable fields include health management apps for young people, chronic disease patient support systems, and remote consultation reservations. Furthermore, by introducing social media-linked scheduling algorithms, technical advances in online behavior-adaptive reservation systems that go beyond mere automation of medical reservation operations by computers are realized.

The proposal unit can estimate the emotions of the patient and adjust the method of proposing online diagnosis based on the estimated emotions of the patient. The proposal unit estimates the emotions of the patient and adjusts the method of proposing online diagnosis based on the estimated emotions. For example, if the patient feels anxious, the proposal unit emphasizes the advantages of online diagnosis in its proposal. If the patient is relaxed, the proposal unit can provide a detailed explanation of online diagnosis. Furthermore, if the patient is in a hurry, the proposal unit can promptly propose online diagnosis. By adjusting the method of proposing online diagnosis according to the patient's emotions, appropriate proposals can be made. Emotion estimation is realized, for example, by using an emotion estimation function such as an emotion engine or generative AI. The generative AI may be a text generation AI (e.g., LLM) or a multimodal generative AI, but is not limited to these examples. Specifically, the proposal unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). The proposal unit vectorizes these input data through preprocessing such as tokenization, spectral analysis, and image feature extraction, and outputs emotion labels (e.g., anxious, relaxed, in a hurry) and confidence scores (0.0-1.0). Examples of AI input include “Text: ‘I am anxious’”, “Voice: tense voice”, “Image: frowning face”; examples of output include “Emotion label: anxious, confidence 0.85”, “Emotion label: relaxed, confidence 0.9”. The proposal unit dynamically switches the algorithm parameters and branching flow of the online diagnosis proposal generation module according to the estimated emotion label. For example, in the case of anxiety, the proposal unit automatically generates proposal sentences that emphasize the advantages of online diagnosis (e.g., no need to visit the hospital, rapid diagnosis, effective use of medical resources); in the case of relaxation, the proposal unit presents detailed explanations of online diagnosis (e.g., diagnostic procedures, privacy protection, communication methods with physicians) step by step; in the case of being in a hurry, the proposal unit immediately generates an online diagnosis reservation form or video call link and presents an interface that allows diagnosis to start with one touch. Examples of AI output include “Input: emotion label: anxious→Output: ‘With online diagnosis, you can receive medical care at home with peace of mind’”, “Input: emotion label: relaxed→Output: ‘Let me explain the flow of online diagnosis’”, “Input: emotion label: in a hurry→Output: ‘You can start online diagnosis immediately’”. The proposal unit displays these outputs on the patient interface, and when the patient selects an option, it registers the online diagnosis in the reservation system and notifies the physician. Unlike conventional uniform proposal sentences or guidance that does not consider emotions, the proposal unit analyzes the patient's emotional state as high-dimensional features and dynamically optimizes the behavior of the proposal generation algorithm, thereby achieving technical effects such as improved patient satisfaction, increased utilization rate of online diagnosis, and efficient use of medical resources. Applicable fields include remote medical services, general outpatient care, and corporate health management. Furthermore, by introducing coordinated control between the emotion estimation AI and proposal generation algorithm and dynamic optimization of parameters, technical advances in emotion-adaptive proposal systems that go beyond mere automation of medical guidance operations by computers are realized.

The proposal unit can refer to the patient's past consultation data when proposing online diagnosis and make the optimal proposal. The proposal unit refers to the patient's past consultation data when proposing online diagnosis and makes the optimal proposal. For example, the proposal unit determines whether online diagnosis is suitable based on the patient's past consultation data and proposes accordingly. The proposal unit can also identify symptoms suitable for online diagnosis from the patient's past consultation data and propose accordingly. Furthermore, the proposal unit can explain the advantages of online diagnosis and propose it by referring to the patient's past consultation data. By referring to the patient's past consultation data, the optimal online diagnosis proposal can be made. Specifically, the proposal unit refers to a consultation history database for each patient (e.g., SQL table, structured data such as patient ID, consultation date, symptoms, diagnosis result, treatment content, online diagnosis usage history, etc.) and extracts past symptom, diagnosis, and treatment patterns in chronological order. The proposal unit extracts features such as cases where online diagnosis was applied (e.g., mild cases, chronic diseases, regular follow-ups), past online diagnosis usage rate, and treatment outcomes after online diagnosis (e.g., revisit rate, satisfaction score) from the history data and inputs them into an online diagnosis proposal AI (e.g., history-adaptive proposal model). Examples of input to the AI include “Patient ID: 12345, last 3 consultations: headache, cough, abdominal pain, online diagnosis usage: ◯, ×, ◯, treatment response: good, normal, good”; the AI vectorizes these history features and dynamically optimizes the applicability and priority of online diagnosis proposals for current symptoms and diagnosis candidates. Examples of output include “Online diagnosis recommendation flag: True, recommendation reason: previous online diagnosis for similar symptoms”, “Online diagnosis not recommended, reason: previous severe case requiring in-person consultation”. The proposal unit displays these outputs on the patient interface, and if online diagnosis is suitable, automatically generates explanations emphasizing its advantages (e.g., no need to visit the hospital, rapid diagnosis) and explanations based on past consultation data (e.g., “Last time, you received appropriate treatment through online diagnosis”). In subsequent processing, when the patient selects online diagnosis, it is registered in the reservation system and recorded in the consultation history database. Unlike conventional uniform proposals or guidance that does not utilize history, the proposal unit analyzes each patient's history data as high-dimensional features and realizes individually optimized online diagnosis proposals, thereby achieving technical effects such as improved proposal accuracy, improved patient satisfaction, and efficient use of medical resources. Applicable fields include chronic disease management clinics, remote medical services, and corporate health management. Furthermore, by introducing time-series analysis of history data and optimization of proposal algorithms, technical advances in history-adaptive proposal systems that go beyond mere automation of medical guidance operations by computers are realized.

The proposal unit can make proposals based on the patient's current health condition and lifestyle habits when proposing online diagnosis. The proposal unit makes proposals based on the patient's current health condition and lifestyle habits when proposing online diagnosis. For example, the proposal unit determines whether online diagnosis is suitable based on the patient's current health condition and proposes accordingly. The proposal unit can also explain the advantages of online diagnosis and propose it by considering the patient's lifestyle habits (e.g., smoking, drinking). Furthermore, the proposal unit can explain the scope of application of online diagnosis and propose it based on the patient's current health condition and lifestyle habits. By considering the patient's health condition and lifestyle habits, appropriate online diagnosis proposals can be made. Specifically, the proposal unit refers to a health condition database for each patient (e.g., chronic disease flag, medication history, medical history, allergy information, etc., structured data) and a lifestyle habits database (e.g., smoking status, drinking frequency, exercise habits, dietary patterns, etc.) and inputs these data into an online diagnosis proposal AI (e.g., health condition and lifestyle habits-adaptive proposal model). Examples of input to the AI include “Medication: antihypertensive, chronic disease: hypertension, lifestyle habits: smoking, no drinking” and “Chronic disease: diabetes, exercise habits: twice a week, diet: vegetable-based”; the AI vectorizes these features and dynamically optimizes the applicability, recommendation level, and explanation content of online diagnosis. Examples of output include “Online diagnosis recommendation flag: True, recommendation reason: suitable for regular follow-up of chronic disease”, “Online diagnosis not recommended, reason: high risk of severe complications”. The proposal unit displays these outputs on the patient interface and automatically generates explanations of the advantages of online diagnosis (e.g., continuity of lifestyle disease management, reduced burden of hospital visits) and scope of application (e.g., “Regular checkups for hypertension can be handled by online diagnosis”) according to the patient's health condition and lifestyle habits. In subsequent processing, when the patient selects online diagnosis, it is registered in the reservation system and recorded in the health condition and lifestyle habits database. Unlike conventional uniform proposals or guidance that does not consider health condition, the proposal unit analyzes each patient's health condition and lifestyle habits as high-dimensional features and realizes individually optimized online diagnosis proposals, thereby achieving technical effects such as improved proposal accuracy, improved patient satisfaction, and efficient use of medical resources. Applicable fields include chronic disease outpatient clinics, lifestyle disease clinics, and remote medical services. Furthermore, by introducing integrated analysis of health condition and lifestyle habits data and optimization of proposal algorithms, technical advances in health-adaptive proposal systems that go beyond mere automation of medical guidance operations by computers are realized.

The proposal unit can estimate the emotions of the patient and determine the priority order of online diagnosis based on the estimated emotions of the patient. The proposal unit estimates the emotions of the patient and determines the priority order of online diagnosis based on the estimated emotions. For example, if the patient feels anxious, the proposal unit prioritizes online diagnosis in its proposals. If the patient is relaxed, the proposal unit can propose online diagnosis according to the patient's preferences. Furthermore, if the patient is in a hurry, the proposal unit can promptly propose online diagnosis. By determining the priority order of online diagnosis according to the patient's emotions, appropriate proposals can be made. Emotion estimation is realized, for example, by using an emotion estimation function such as an emotion engine or generative AI. The generative AI may be a text generation AI (e.g., LLM) or a multimodal generative AI, but is not limited to these examples. Specifically, the proposal unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). The proposal unit vectorizes these inputs through preprocessing such as tokenization, spectral analysis, and image feature extraction, and outputs emotion labels (e.g., anxious, relaxed, in a hurry) and confidence scores (0.0-1.0). Examples of AI input include “Text: ‘I am anxious’”, “Voice: tense voice”, “Image: frowning face”; examples of output include “Emotion label: anxious, confidence 0.85”, “Emotion label: relaxed, confidence 0.9”. The proposal unit dynamically adjusts the order and recommendation scores of the online diagnosis proposal candidate list according to the estimated emotion label. For example, in the case of anxiety, online diagnosis is displayed at the top; in the case of relaxation, proposals that emphasize the patient's preferences and detailed explanations are placed at the top; in the case of being in a hurry, online diagnosis slots that can be started immediately are presented at the top. Examples of AI output include “Input: emotion label: anxious→Output: online diagnosis priority: high”, “Input: emotion label: relaxed→Output: online diagnosis priority: medium”. In subsequent processing, the diagnostic method selected by the patient is registered in the reservation system and reflected in optimization of the emotion-adaptive interface. Unlike conventional uniform proposal order or guidance that does not consider emotions, the proposal unit analyzes the patient's emotional state as high-dimensional features and realizes individually optimized online diagnosis proposals, thereby achieving technical effects such as improved patient satisfaction, increased utilization rate of online diagnosis, and efficient use of medical resources. Applicable fields include remote medical services, general outpatient care, and corporate health management. Furthermore, by introducing coordinated control between the emotion estimation AI and proposal candidate generation algorithm and dynamic optimization of parameters, technical advances in emotion-adaptive proposal systems that go beyond mere automation of medical guidance operations by computers are realized.

The proposal unit can make appropriate proposals based on the patient's geographic location information when proposing online diagnosis. The proposal unit makes optimal proposals by considering the patient's geographic location information when proposing online diagnosis. For example, the proposal unit proposes online diagnosis by considering the medical resources available in the patient's residential area. The proposal unit can also propose online diagnosis when commuting is difficult based on the patient's geographic location information. Furthermore, the proposal unit can explain the advantages of online diagnosis and propose it by considering the patient's geographic location information. By considering the patient's geographic location information, appropriate online diagnosis proposals can be made. Specifically, the proposal unit integrally refers to the patient's current location information (e.g., GPS coordinates, address data), residential area label (e.g., prefecture, city/ward/town/village), and regional medical resource database (e.g., number of medical institutions, medical departments, access difficulty, transportation information, etc.). The proposal unit inputs this information into a geographic information-linked online diagnosis proposal AI (e.g., geographic information system-linked proposal model) and automatically extracts the patient's difficulty in commuting and the status of regional medical resources. Examples of input to the AI include “Location: Shinjuku, Tokyo, number of medical institutions: many, transportation: good”, “Location: rural Hokkaido, number of medical institutions: few, transportation: inconvenient”; the AI vectorizes these geographic features and dynamically optimizes the applicability, recommendation level, and explanation content of online diagnosis proposals. Examples of output include “Online diagnosis recommendation flag: True, recommendation reason: region with difficulty in commuting”, “Online diagnosis recommendation flag: False, reason: many specialists nearby”. The proposal unit displays these outputs on the patient interface and automatically generates explanations of the advantages of online diagnosis (e.g., receiving medical care from remote locations, saving transportation costs and time) and scope of application (e.g., “Online diagnosis is convenient in your area”) according to the patient's geographic location information. In subsequent processing, when the patient selects online diagnosis, it is registered in the reservation system and recorded in the regional medical resource database. Unlike conventional uniform proposals or guidance that does not consider geographic information, the proposal unit analyzes the patient's geographic location information as high-dimensional features and realizes individually optimized online diagnosis proposals, thereby achieving technical effects such as reduced commuting burden, increased utilization rate of online diagnosis, and improved patient satisfaction. Applicable fields include remote consultation in wide-area medical zones, guidance for local medical institutions, and health management systems for travelers. Furthermore, by introducing geographic information-linked proposal algorithms, technical advances in geographic-adaptive proposal systems that go beyond mere automation of medical guidance operations by computers are realized.

The proposal unit can analyze the patient's social media activity when proposing online diagnosis and make the optimal proposal. The proposal unit analyzes the patient's social media activity when proposing online diagnosis and makes the optimal proposal. For example, the proposal unit determines whether online diagnosis is suitable based on the patient's social media activity and proposes accordingly. The proposal unit can also explain the advantages of online diagnosis and propose it by considering the patient's social media activity. Furthermore, the proposal unit can explain the scope of application of online diagnosis and propose it by analyzing the patient's social media activity. By analyzing the patient's social media activity, appropriate online diagnosis proposals can be made. Specifically, with the patient's consent, the proposal unit obtains the patient's posting history (e.g., health-related post text, images, videos), followed account list (e.g., medical institutions, health information providers), and group participation information (e.g., chronic disease patient associations, health support groups) via social media APIs. The proposal unit inputs these data into a natural language processing AI (e.g., BERT-based health-related text classification model, multimodal post analysis model) and extracts health status, symptoms of interest, and interest level in online diagnosis from post content and following tendencies. Examples of AI input include “Post text: ‘Recently used online diagnosis’, ‘Visiting the hospital is difficult’”, “Followed account: remote medical service”, “Group participation: online diagnosis experience group”; the AI vectorizes these features and dynamically optimizes the applicability, recommendation level, and explanation content of online diagnosis proposals. Examples of output include “Online diagnosis recommendation flag: True, recommendation reason: experience using online diagnosis”, “Online diagnosis recommendation flag: False, reason: tendency to prefer face-to-face consultation”. The proposal unit displays these outputs on the patient interface and automatically generates explanations of the advantages of online diagnosis (e.g., testimonials from users, convenience) and scope of application (e.g., “Many people who have used online diagnosis are satisfied”) according to the patient's social media activity. In subsequent processing, when the patient selects online diagnosis, it is registered in the reservation system and recorded in the online activity database. Unlike conventional uniform proposals or guidance that does not utilize social media, the proposal unit analyzes the patient's online activity data as high-dimensional features and realizes individually optimized online diagnosis proposals, thereby achieving technical effects such as improved proposal accuracy, improved patient satisfaction, and increased utilization rate of online diagnosis. Applicable fields include health management apps for young people, chronic disease patient support systems, and remote medical services. Furthermore, by introducing social media-linked proposal algorithms, technical advances in online behavior-adaptive proposal systems that go beyond mere automation of medical guidance operations by computers are realized.

The system according to the embodiment is not limited to the examples described above and can be variously modified, for example, as follows. Specifically, the system has extensibility through modularization of each component and API integration, and each function of the reception unit, analysis unit, determination unit, and proposal unit can be implemented as independent microservices. The system can be applied to cloud-based distributed processing environments and hybrid configurations linked with edge devices, and various interfaces can be used to acquire patient data, such as in-hospital terminals, smartphone apps, wearable devices, and remote medical terminals. The system can also flexibly change the types and training methods of AI models; for example, as the symptom analysis AI, not only transformer-based large language models but also CNNs, RNNs, graph neural networks, self-supervised learning models, and transfer learning models can be used in combination. The database configuration can also be selected according to the application, such as relational databases, NoSQL databases, time-series databases, and knowledge graphs. Furthermore, security functions such as data encryption, anonymization, access control, and audit log recording can be additionally implemented according to patient privacy protection and security requirements. The input/output specifications of the AI can also be extended; for example, voice recognition AI, image analysis AI, and vital sensor data analysis AI can be added to improve analysis accuracy through multimodal data integration. Subsequent processing can be linked not only to consultation reservations but also to various medical-related services such as medication guidance, health management proposals, rehabilitation plan generation, and medical cost estimation. Unlike conventional single-function systems or simple automation of human tasks, the present system demonstrates technical effects that enable adaptation to diverse needs and operational environments in medical settings through diverse combinations of AI models and flexible data flow design. Applicable fields include general outpatient care, emergency medical care, remote medical care, occupational health, corporate health management, home medical care, and nursing care support systems. Furthermore, by introducing extensibility, flexibility, enhanced security, and multimodal AI integration, the system realizes advances in computer technology itself and accelerates medical DX.

The reception unit can refer to the patient's past consultation data when inputting symptoms and supplement the input content. For example, symptoms frequently reported by the patient in the past are automatically displayed as candidates. The reception unit can also automatically generate questions related to specific symptoms from the patient's past consultation data and present them to the patient. Furthermore, the reception unit can propose additional tests or questions for specific symptoms based on the patient's past consultation data. By utilizing the patient's past consultation data, the accuracy and efficiency of symptom input can be improved. Specifically, the reception unit refers to a consultation history database for each patient (e.g., SQL table, structured data such as patient ID, consultation date, symptoms, diagnosis result, treatment content, test results, etc.) and extracts past symptom input history and diagnosis patterns in chronological order. The reception unit extracts features such as frequently reported symptoms (e.g., text labels such as “headache,” “cough,” top 3 by frequency), past diagnosis results (e.g., ICD-10 codes, severity scores), and test history (e.g., presence or absence of blood tests, imaging tests) from the history data and inputs them into a symptom input completion AI (e.g., history-adaptive candidate generation model, automatic question generation model). Examples of input to the AI include “Patient ID: 12345, last 3 symptoms: headache, cough, headache; past diagnoses: migraine, cold, migraine; test history: blood test, imaging test, blood test”; the AI vectorizes these history features and automatically generates a candidate symptom list for the current symptom input screen (e.g., “headache (0.8), cough (0.6)”), related question list (e.g., “How often do you have headaches?”, “How long have you had a cough?”), and additional test proposals (e.g., “Blood test recommended”). Examples of output include “Candidate symptoms: headache (0.8), cough (0.6)”, “Related question: How often do you have headaches?”, “Additional test: blood test recommended”. The reception unit presents these outputs as candidate buttons or question forms on the user interface, allowing the patient to select or answer with one touch. In subsequent processing, the content selected or answered by the patient is recorded in the consultation database and reflected in candidate generation and automatic question generation for future appointments. Unlike conventional uniform input screens or reception systems that do not utilize history, the reception unit analyzes each patient's history data as high-dimensional features and provides individually optimized input experiences and question generation, thereby achieving technical effects such as greatly improved input efficiency, reduced input errors, and improved diagnostic accuracy. Applicable fields include general outpatient reception, chronic disease management clinics, remote medical reception, and health management systems. Furthermore, by introducing time-series analysis of history data and automatic question generation algorithms, technical advances in history-adaptive reception systems that go beyond mere automation of reception operations by computers are realized.

The analysis unit can refer to the patient's lifestyle habits data when analyzing symptoms and improve the accuracy of the analysis. For example, the analysis unit prioritizes the analysis of symptoms related to the patient's smoking or drinking habits. The analysis unit can also evaluate the risk of specific symptoms based on the patient's exercise habits and diet. Furthermore, the analysis unit can propose preventive measures or lifestyle improvements for specific symptoms based on the patient's lifestyle habits data. By utilizing the patient's lifestyle habits data, the accuracy and usefulness of symptom analysis can be improved. Specifically, the analysis unit refers to a lifestyle habits database for each patient (e.g., smoking status, drinking frequency, exercise habits, dietary patterns, sleep duration, stress level, etc., structured data) and inputs these data into a symptom analysis AI (e.g., lifestyle habits-adaptive multimodal model). Examples of input to the AI include “Smoking: yes, drinking: 3 times a week, exercise: once a week, diet: high fat, low vegetables”; “Smoking: no, drinking: none, exercise: 3 times a week, diet: vegetable-based”; the AI vectorizes these lifestyle features and reflects them in the symptom analysis task (e.g., candidate disease list generation, risk factor estimation, severity scoring, preventive measure proposal). Examples of output include “Candidate diseases: COPD (0.7), fatty liver (0.5), hypertension (0.4)”, “Risk factors: smoking (0.8), drinking (0.6)”, “Preventive measures: recommend quitting smoking, improving exercise habits”. Furthermore, based on lifestyle habits data, the analysis unit optimizes the analysis algorithm by weighting specific risk factors (e.g., risk of respiratory disease due to smoking, risk of liver disease due to drinking, risk of lifestyle disease due to lack of exercise) and automatically generates optimal analysis results and preventive/lifestyle improvement proposals for each patient. In subsequent processing, analysis results and proposals are recorded in the consultation database and used for explanatory materials for physicians and health guidance for patients. Unlike conventional uniform analysis flows or analysis systems that do not consider lifestyle habits, the analysis unit analyzes each patient's lifestyle habits data as high-dimensional features and dynamically optimizes the behavior of the analysis AI, thereby achieving technical effects such as improved analysis accuracy, reduced misdiagnosis rate, and promotion of preventive medicine. Applicable fields include lifestyle disease outpatient clinics, health checkup systems, remote medical diagnosis, and corporate health management. Furthermore, by introducing integrated analysis of lifestyle habits data and automatic preventive measure proposal algorithms, technical advances in lifestyle habits-adaptive analysis systems that go beyond mere automation of medical analysis operations by computers are realized.

The determination unit can propose the optimal consultation date and time by considering the patient's occupational information when determining the consultation date and time. For example, if the patient works in shifts, the determination unit proposes consultation dates and times that match the work shift. If the patient has a job that involves business trips or long working hours, the determination unit can propose consultation dates and times by considering the business trip schedule and working hours. Furthermore, the determination unit can propose dates and times that are convenient for consultation based on the patient's occupational information. By considering the patient's occupational information, the optimal consultation date and time can be proposed. Specifically, the determination unit refers to an occupational information database for each patient (e.g., occupation code, work style, shift pattern, business trip schedule, working hours, holiday information, etc., structured data) and inputs this information into an occupation-adaptive consultation date and time determination AI (e.g., scheduling model+constraint satisfaction problem solver). Examples of input to the AI include “Occupation: nurse, work style: three-shift system, next shift: night shift on 7/22”; “Occupation: sales, business trip schedule: 7/23-7/25, working hours: 9:00-18:00”; the AI vectorizes these occupational features and outputs a list of candidate consultation dates and times (e.g., 7/22 14:00, 7/26 10:00) and recommendation scores (0.9, 0.8). Furthermore, based on occupational information, the determination unit preferentially displays candidate dates and times that consider holidays and intervals between shifts for shift workers, and applies an algorithm that automatically excludes business trip periods when there is a business trip schedule. Examples of output include “Candidate dates and times: 7/22 14:00 (0.9), 7/26 10:00 (0.8), recommended outside working hours”. In subsequent processing, the date and time selected by the patient is registered in the reservation system and recorded in the occupational information database. Unlike conventional uniform date and time proposals or reservation systems that do not consider occupation, the determination unit analyzes each patient's occupational information as high-dimensional features and realizes individually optimized consultation date and time proposals, thereby achieving technical effects such as improved patient satisfaction, reduced reservation cancellation rates, and increased efficiency of medical care. Applicable fields include outpatient reservation management, clinics for shift workers, remote consultation reservations, and occupational health checkups. Furthermore, by introducing occupation-linked scheduling algorithms, technical advances in occupation-adaptive reservation systems that go beyond mere automation of medical reservation operations by computers are realized.

The proposal unit can customize the content of proposals for online diagnosis based on the patient's current health condition and lifestyle habits. For example, if the patient has a chronic disease, the proposal unit prioritizes proposals for online diagnosis related to that disease. The proposal unit can also propose online diagnosis for health risks related to the patient's lifestyle habits (e.g., smoking, drinking). Furthermore, the proposal unit can propose online diagnosis that includes specific health improvement measures or preventive measures based on the patient's current health condition and lifestyle habits. By proposing the optimal online diagnosis based on the patient's health condition and lifestyle habits, appropriate proposals can be made. Specifically, the proposal unit refers to a health condition database for each patient (e.g., chronic disease flag, medication history, medical history, allergy information, etc., structured data) and a lifestyle habits database (e.g., smoking status, drinking frequency, exercise habits, dietary patterns, etc.) and inputs these data into an online diagnosis proposal AI (e.g., health condition and lifestyle habits-adaptive proposal model). Examples of input to the AI include “Medication: antihypertensive, chronic disease: hypertension, lifestyle habits: smoking, no drinking” and “Chronic disease: diabetes, exercise habits: twice a week, diet: vegetable-based”; the AI vectorizes these features and dynamically optimizes the applicability, recommendation level, explanation content, health improvement measures, and preventive measures for online diagnosis. Examples of output include “Online diagnosis recommendation flag: True, recommendation reason: suitable for regular follow-up of chronic disease, health improvement measure: recommend quitting smoking”; “Online diagnosis not recommended, reason: high risk of severe complications”. The proposal unit displays these outputs on the patient interface and automatically generates explanations of the advantages of online diagnosis (e.g., continuity of lifestyle disease management, reduced burden of hospital visits), scope of application (e.g., “Regular checkups for hypertension can be handled by online diagnosis”), and health improvement measures or preventive measures (e.g., “Continue to quit smoking”, “Increase exercise habits”) according to the patient's health condition and lifestyle habits. In subsequent processing, when the patient selects online diagnosis, it is registered in the reservation system and recorded in the health condition and lifestyle habits database. Unlike conventional uniform proposals or guidance that does not consider health condition, the proposal unit analyzes each patient's health condition and lifestyle habits as high-dimensional features and realizes individually optimized online diagnosis proposals, thereby achieving technical effects such as improved proposal accuracy, improved patient satisfaction, and efficient use of medical resources. Applicable fields include chronic disease outpatient clinics, lifestyle disease clinics, remote medical services, and health management apps. Furthermore, by introducing integrated analysis of health condition and lifestyle habits data and automatic health improvement measure proposal algorithms, technical advances in health-adaptive proposal systems that go beyond mere automation of medical guidance operations by computers are realized.

The reception unit can estimate the emotions of the patient and provide support for symptom input based on the estimated emotions of the patient. For example, if the patient feels anxious, the reception unit displays gentle words or encouraging messages to provide reassurance. If the patient is relaxed, the reception unit can provide detailed input options to enable more accurate information input. Furthermore, if the patient is in a hurry, the reception unit can provide concise and quick input methods to reduce stress. By providing support for symptom input according to the patient's emotions, the accuracy and efficiency of input can be improved. Specifically, the reception unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model, multimodal emotion estimation model). Examples of AI input include “Text: ‘I am anxious’”, “Voice: tense voice”, “Image: frowning face”; the AI vectorizes these inputs through preprocessing such as tokenization, spectral analysis, and image feature extraction, and outputs emotion labels (e.g., anxious, relaxed, in a hurry) and confidence scores (0.0-1.0). Examples of output include “Emotion label: anxious, confidence 0.85”, “Emotion label: relaxed, confidence 0.9”. The reception unit dynamically switches the display content and input procedure of the symptom input support module according to the estimated emotion label. For example, in the case of anxiety, the reception unit displays encouragement messages and input guides in large font and minimizes the number of input items. In the case of relaxation, the reception unit adds detailed input options and supplementary explanations to encourage accurate information input. In the case of being in a hurry, the reception unit prioritizes shortcut buttons for quick input of main symptoms and voice input. In subsequent processing, the content input by the patient is recorded in the consultation database and reflected in optimization of the emotion-adaptive interface and generation of support content for future appointments. Unlike conventional uniform input support or reception systems that do not consider emotions, the reception unit analyzes the patient's emotional state as high-dimensional features and provides individually optimized input support, thereby achieving technical effects such as improved input efficiency, reduced input errors, and improved patient satisfaction. Applicable fields include medical reception terminals, remote medical apps, and health management apps. Furthermore, by introducing coordinated control between the emotion estimation AI and input support generation algorithm and dynamic optimization of parameters, technical advances in emotion-adaptive reception systems that go beyond mere automation of reception operations by computers are realized.

The analysis unit can estimate the emotions of the patient and adjust the priority order of symptom analysis based on the estimated emotions of the patient. For example, if the patient feels anxious, the analysis unit prioritizes the analysis of urgent symptoms. If the patient is relaxed, the analysis unit can perform detailed symptom analysis. Furthermore, if the patient is in a hurry, the analysis unit can quickly analyze the main symptoms. By adjusting the priority order of symptom analysis according to the patient's emotions, appropriate analysis can be performed. Specifically, the analysis unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model, multimodal emotion estimation model). Examples of AI input include “Text: ‘I am anxious’”, “Voice: tense voice”, “Image: frowning face”; the AI vectorizes these inputs through preprocessing such as tokenization, spectral analysis, and image feature extraction, and outputs emotion labels (e.g., anxious, relaxed, in a hurry) and confidence scores (0.0-1.0). Examples of output include “Emotion label: anxious, confidence 0.85”, “Emotion label: relaxed, confidence 0.9”. The analysis unit dynamically switches the parameters and inference flow of the symptom analysis AI (e.g., transformer-based large language model or multimodal model) according to the estimated emotion label. For example, in the case of anxiety, the weight of the urgency estimation task is increased, urgent symptoms (e.g., cerebral hemorrhage, severe infection) are prioritized for analysis, and detailed symptom classification is omitted. In the case of relaxation, the top five candidate diseases are analyzed in detail, and severity scores and supplementary risk factors are also output. In the case of being in a hurry, only the main symptoms are analyzed quickly, and a simple candidate disease list (top 1-2 items) and urgency score are output. Examples of AI output include “Input: ‘I am anxious’+headache→Output: urgency 0.9, candidate diseases: cerebral hemorrhage (0.7), migraine (0.2)”, “Input: ‘I am relaxed’+cough→Output: urgency 0.3, candidate diseases: cold (0.6), bronchitis (0.3), pneumonia (0.1)”. In subsequent processing, if urgency is high, an immediate consultation slot is preferentially assigned; if low, online diagnosis or later reservation is proposed. Unlike conventional uniform analysis flows or subjective human judgment, the analysis unit analyzes the patient's emotional state as high-dimensional features and dynamically optimizes the behavior of the analysis AI, thereby achieving technical effects such as improved analysis accuracy, reduced misdiagnosis rate, and improved patient experience. Applicable fields include emergency outpatient triage, remote medical diagnosis, and health management systems. Furthermore, by introducing coordinated control between the emotion estimation AI and symptom analysis AI and dynamic optimization algorithms for parameters, technical advances in emotion-adaptive analysis systems that go beyond mere automation of medical analysis operations by computers are realized.

The determination unit can adjust the method of proposing consultation dates and times based on the estimated emotions of the patient. For example, if the patient feels anxious, the determination unit preferentially proposes an earlier consultation date and time. If the patient is relaxed, the determination unit can propose a consultation date and time that matches the patient's preferences. Furthermore, if the patient is in a hurry, the determination unit can propose the earliest possible consultation date and time. By adjusting the method of proposing consultation dates and times according to the patient's emotions, appropriate consultation dates and times can be proposed. Specifically, the determination unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). Examples of AI input include “Text: ‘I am anxious’”, “Voice: tense voice”, “Image: frowning face”; the AI vectorizes these inputs through preprocessing such as tokenization, spectral analysis, and image feature extraction, and outputs emotion labels (e.g., anxious, relaxed, in a hurry) and confidence scores (0.0-1.0). Examples of output include “Emotion label: anxious, confidence 0.85”, “Emotion label: relaxed, confidence 0.9”. The determination unit dynamically switches the parameters and branching flow of the consultation date and time determination algorithm (e.g., priority scheduling, constraint satisfaction problem solver) according to the estimated emotion label. For example, in the case of anxiety, the weights for urgency and early consultation preference are increased, and the shortest available slots are preferentially extracted from the reservation management database (SQL table, reservation ID, date and time, attending physician, availability, etc.), generating a candidate date and time list (e.g., 2024/07/01 09:00, 2024/07/01 10:00) and recommendation scores (0.95, 0.85). In the case of relaxation, the patient's desired date and time (calendar format, desired date list) is emphasized, and the date and time closest to the preference is proposed. In the case of being in a hurry, immediate consultation slots or waiting list slots are automatically searched, and only the earliest date and time is presented. Examples of AI output include “Input: emotion label: anxious+desired date and time: morning of 7/1→Output: candidate date and time: 7/1 09:00 (0.95), 7/1 10:00 (0.85)”, “Input: emotion label: in a hurry→Output: candidate date and time: today 15:00 (0.99)”. In subsequent processing, when the patient selects a proposed date and time, that date and time is registered in the reservation system and notified to the physician. Unlike conventional uniform date and time determination or subjective human judgment, the determination unit analyzes the patient's emotional state as high-dimensional features in cooperation with the analysis AI and dynamically optimizes the behavior of the consultation date and time determination algorithm, thereby achieving technical effects such as improved patient satisfaction, reduced waiting times, and increased efficiency of medical care. Applicable fields include outpatient reservation management, emergency consultation scheduling, and remote consultation reservations. Furthermore, by introducing coordinated control between the emotion estimation AI and the consultation date and time determination algorithm and dynamic optimization of parameters, technical advances in emotion-adaptive reservation systems that go beyond mere automation of medical reservation operations by computers are realized.

The proposal unit is capable of estimating the emotions of the patient and adjusting the content of the online diagnosis proposal based on the estimated emotions. For example, if the patient feels anxious, the advantages of online diagnosis are emphasized in the proposal. If the patient is relaxed, a detailed explanation of the online diagnosis may be provided. Furthermore, if the patient is in a hurry, the online diagnosis can be proposed promptly. By adjusting the content of the online diagnosis proposal according to the patient's emotions, appropriate proposals can be made. Specifically, the proposal unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). The proposal unit vectorizes these input data through preprocessing such as tokenization, spectral analysis, and image feature extraction, and outputs emotion labels (e.g., anxious, relaxed, in a hurry) and confidence scores (0.0-1.0). Examples of AI input include “Text: ‘I am anxious’”, “Voice: tense voice”, “Image: frowning face”; examples of output include “Emotion label: anxious, confidence 0.85”, “Emotion label: relaxed, confidence 0.9”. The proposal unit dynamically switches the algorithm parameters and branching flow of the online diagnosis proposal generation module according to the estimated emotion label. For example, in the case of anxiety, a proposal sentence emphasizing the advantages of online diagnosis (e.g., no need to visit the hospital, rapid diagnosis, effective use of medical resources) is automatically generated; in the case of relaxation, a step-by-step presentation of detailed explanations of online diagnosis (e.g., diagnostic procedures, privacy protection, communication methods with doctors) is provided; in the case of being in a hurry, an online diagnosis reservation form or video call link is generated immediately, and an interface for one-touch diagnosis initiation is presented. Examples of AI output include “Input: Emotion label: anxious→Output: ‘With online diagnosis, you can receive medical care at home with peace of mind’”, “Input: Emotion label: relaxed→Output: ‘Let me explain the flow of online diagnosis’”, “Input: Emotion label: in a hurry→Output: ‘You can start online diagnosis immediately’”. The proposal unit displays these outputs on the patient interface, and if the patient selects them, registers them in the online diagnosis reservation system and notifies the physician. Unlike conventional uniform proposal sentences or emotion-agnostic guidance, the proposal unit analyzes the patient's emotional state as high-dimensional feature quantities and dynamically optimizes the behavior of the proposal generation algorithm, thereby achieving technical effects such as improved patient satisfaction, increased online diagnosis utilization rate, and efficient use of medical resources. Applicable fields include telemedicine services, general outpatient care, and corporate health management. Furthermore, by introducing coordinated control between emotion estimation AI and proposal generation algorithms and dynamic parameter optimization, the technical advancement of an emotion-adaptive proposal system is realized, surpassing mere automation of medical guidance operations by computers.

The reception unit is capable of estimating the emotions of the patient and adjusting the symptom input interface based on the estimated emotions. For example, if the patient feels anxious, a simple and intuitive interface is provided, minimizing the input steps. If the patient is relaxed, detailed input options are provided, and customizable input methods may be proposed. Furthermore, if the patient is in a hurry, voice input is prioritized to enable rapid symptom entry. By adjusting the interface according to the patient's emotions, symptom input can be performed smoothly. Specifically, the reception unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model or multimodal emotion estimation model). Examples of AI input include “Text: ‘I am anxious’”, “Voice: tense voice”, “Image: frowning face”; the AI vectorizes these inputs through preprocessing such as tokenization, spectral analysis, and image feature extraction, and outputs emotion labels (e.g., anxious, relaxed, in a hurry) and confidence scores (0.0-1.0). Examples of output include “Emotion label: anxious, confidence 0.85”, “Emotion label: relaxed, confidence 0.9”. The reception unit dynamically switches the layout, input steps, and input methods (e.g., number of buttons, number of input fields, prioritized display of voice input) of the interface generation module according to the estimated emotion label. For example, in the case of anxiety, the number of buttons is reduced, encouraging messages are displayed, and input steps are minimized. In the case of relaxation, detailed input options and customizable input methods are additionally displayed. In the case of being in a hurry, voice input is prioritized, and only the main symptoms can be entered simply. As a subsequent process, the content entered by the patient is recorded in the consultation database and reflected in the optimization of the emotion-adaptive interface and the layout generation for subsequent visits. Unlike conventional uniform input screens or emotion-agnostic reception systems, the reception unit analyzes the patient's emotional state as high-dimensional feature quantities and provides individually optimized interfaces, thereby achieving technical effects such as improved input efficiency, reduced input errors, and improved patient satisfaction. Applicable fields include medical reception terminals, telemedicine apps, and health management apps. Furthermore, by introducing coordinated control between emotion estimation AI and interface generation algorithms and dynamic parameter optimization, the technical advancement of an emotion-adaptive reception system is realized, surpassing mere automation of reception operations by computers.

The analysis unit is capable of estimating the emotions of the patient and adjusting the display method of the analysis results based on the estimated emotions. For example, if the patient feels anxious, a simple and highly visible display method is provided. If the patient is relaxed, a display method including detailed information may be provided. Furthermore, if the patient is in a hurry, a display method focusing on key points may be provided. By adjusting the display method of the analysis results according to the patient's emotions, appropriate information can be provided. Specifically, the analysis unit inputs the patient's text input (e.g., “I am anxious,” “I am relaxed,” “I am in a hurry,” up to 256 tokens), voice data (16 kHz, 16 bit, WAV format, tone and speed of voice), and image data (facial expression image, RGB image tensor: 224×224×3) into an emotion estimation AI (e.g., BERT-based emotion classification model, multimodal emotion estimation model), and outputs emotion labels (e.g., anxious, relaxed, in a hurry) and confidence scores. Examples of AI input include “Text: ‘I am anxious’”, “Voice: tense voice”, “Image: frowning face”; examples of output include “Emotion label: anxious, confidence 0.85”, “Emotion label: relaxed, confidence 0.9”. The analysis unit dynamically switches the layout, amount of information, and highlighted items of the analysis result display module according to the estimated emotion label. For example, in the case of anxiety, only the main diagnosis result and urgency are displayed simply in large font, and detailed information is hidden. In the case of relaxation, detailed information such as candidate disease lists, severity scores, risk factors, and recommended treatments are provided in tab format or expandable display. In the case of being in a hurry, only the key points (e.g., the most suspected disease name and urgency score) are displayed concisely, and the operation steps are minimized. As a subsequent process, if the patient requests detailed information, additional information is displayed stepwise, and the display history is recorded in the consultation database. Unlike conventional uniform display screens or emotion-agnostic information provision, the analysis unit analyzes the patient's emotional state as high-dimensional feature quantities and dynamically optimizes the behavior of the analysis result display module, thereby achieving technical effects such as improved efficiency of information transmission, improved patient satisfaction, and reduced misunderstanding and anxiety. Applicable fields include medical reception terminals, telemedicine apps, and health management apps. Furthermore, by coordinating emotion estimation AI and display control algorithms, the technical advancement of an emotion-adaptive information provision system is realized, surpassing mere automation of medical information provision operations by computers.

The following is a brief explanation of the processing flow of Example of the Embodiment. Specifically, the present system integrates the modules of the reception unit, analysis unit, determination unit, and proposal unit to automate the entire process from patient data acquisition to diagnosis, reservation, and proposal. The reception unit accepts patient symptom input and attribute information (e.g., age, gender, medical history, lifestyle habits, emotional state, etc.) through various interfaces (tablet terminals, smartphone apps, voice input, image input, etc.). The analysis unit uses transformer-based large language models, multimodal models, and image analysis AI (such as CNN) to perform integrated analysis of input symptoms, image, and voice data as high-dimensional feature quantities, and outputs candidate disease lists, urgency scores, severity scores, risk factors, and so on. The determination unit integrates the output from the analysis unit with the patient's desired date and time, reservation management database, occupation, living situation, geographic information, etc., and uses priority-based scheduling algorithms and constraint satisfaction problem solvers to generate an optimal list of consultation date and time candidates and recommendation scores. The proposal unit automatically generates and presents online diagnosis, health management proposals, preventive measures, lifestyle improvement suggestions, etc., based on the diagnosis results, patient's emotions, health condition, lifestyle habits, geographic information, online activity data, and so on, in the most suitable form for the patient interface. The modules communicate asynchronously via API integration and message queues, and the database is updated in real time. Unlike conventional manual operations or simple automation, the present system achieves significant improvements in diagnostic accuracy, reservation optimization, and patient experience through multi-layered AI collaboration and high-dimensional feature analysis. Applicable fields include outpatient care, emergency triage, telemedicine, health management systems, occupational health, and home medical care. Furthermore, the introduction of multimodal AI collaboration and data-driven algorithms realizes advances in computer technology itself and accelerates medical DX.

Step 1: The reception unit inputs symptoms when the patient visits the hospital. The patient only needs to easily enter their symptoms, such as “I have a headache” or “I have a cough.” This information is input into the generative AI. Step 2: The analysis unit analyzes the entered symptoms using the generative AI. The generative AI analyzes the patient's symptoms and, in cooperation with the image analysis AI, narrows down the patient's symptoms to some extent. For example, if the patient has a headache, the image analysis AI analyzes the patient's complexion and facial expression to determine whether the symptoms are urgent. This enables rapid narrowing down of the patient's symptoms. Step 3: The determination unit determines the optimal consultation date and time based on the narrowed-down symptoms, existing reservations, and the patient's desired date and time. For example, if the patient wishes to have a consultation in the morning of the next day, the generative AI checks the existing reservation status and can propose the optimal consultation date and time, thereby shortening the patient's waiting time. Step 4: The proposal unit can switch to online diagnosis depending on the diagnosis result by the generative AI. For example, if the symptoms are mild and it is determined that online diagnosis is possible, the generative AI can propose online diagnosis to the patient. This eliminates the need for the patient to visit the hospital and enables efficient consultation. Specifically, in Step 1, the reception unit receives multimodal data such as the patient's text input (e.g., “I have a headache,” “I have a cough,” up to 256 tokens), voice data (16 kHz, 16 bit, WAV format), and image data (face photo, affected area image, RGB image tensor: 224×224×3), performs noise removal and normalization in the preprocessing unit, and inputs them into the generative AI (transformer-based large language model or multimodal model). In Step 2, the analysis unit tokenizes the input text, vectorizes it using self-attention mechanisms, and performs symptom classification (e.g., ICD-10 code classification, severity scoring) and urgency estimation (probability value from 0 to 1, judged as urgent if threshold is 0.7 or higher). The image analysis AI uses CNN architectures such as ResNet or EfficientNet to analyze complexion (RGB histogram, blood color estimation) and facial expression (facial muscle feature point extraction, emotion estimation), and feeds auxiliary features for urgency and severity back to the generative AI. The analysis unit integrates these multimodal features and outputs a candidate disease list (e.g., migraine, tension-type headache, cerebral hemorrhage) and probability distribution for each disease. An example of AI input/output is “Input: ‘I have a headache’+face image→Output: ‘Urgency 0.2, candidate diseases: migraine (0.7), tension-type headache (0.2), cerebral hemorrhage (0.1)’”. In Step 3, the determination unit receives the candidate disease list and urgency score output from the analysis unit (e.g., urgency 0.8, candidate diseases: pneumonia (0.6), bronchitis (0.3), common cold (0.1)), matches them with the hospital's reservation management database (SQL table, reservation ID, date and time, attending physician, availability, etc.) and the patient's desired date and time (calendar format, desired date list). The determination unit uses combinatorial optimization algorithms (e.g., constraint satisfaction problems, priority-based scheduling) to calculate the optimal consultation date and time that simultaneously satisfies multiple constraints such as urgency, patient preferences, and physician availability. Examples of AI input include “candidate disease list+urgency+desired date and time+reservation database”; examples of output include “date and time candidate list (2024/07/01 10:00, 2024/07/01 11:30, etc.)+recommendation scores (0.9, 0.7, etc.)”. In Step 4, the proposal unit automatically generates proposals for switching to online diagnosis based on the diagnosis result by the generative AI (e.g., mild/moderate/severe label, urgency score, online diagnosis availability flag). Examples of AI input include “diagnosis result label+urgency+patient attribute information”; examples of output include “online diagnosis recommendation flag (True/False), video call link generation, online diagnosis reservation form auto-generation,” etc. As a subsequent process, if the patient selects online diagnosis, it is registered in the reservation system and notified to the physician. Unlike conventional interviews and visual judgments by humans, the present system greatly improves consistency, objectivity, and speed of judgment through statistical and machine learning analysis in high-dimensional space and multi-layered AI collaboration. Technical effects include improved accuracy of symptom analysis, reduced misdiagnosis rate, faster diagnosis, shorter waiting times, improved consultation efficiency, improved patient satisfaction, and effective use of medical resources. Applicable fields include outpatient care, emergency triage, telemedicine, and health management systems.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, analysis unit, determination unit, and proposal unit is implemented by at least one of, for example, a smart device 14 and a data processing device 12. For example, the reception unit is implemented by a reception device 38 of the smart device 14, allowing a patient to input symptoms. The analysis unit is implemented, for example, by a specific processing unit 290 of the data processing device 12, and analyzes the input symptoms using a generative AI. The determination unit is implemented, for example, by the specific processing unit 290 of the data processing device 12, and determines the optimal consultation date and time based on the narrowed-down symptoms. The proposal unit is implemented, for example, by a control unit 46A of the smart device 14, and proposes an online diagnosis. The correspondence between each unit and the device or control unit is not limited to the examples described above and can be variously modified.

### Second Embodiment

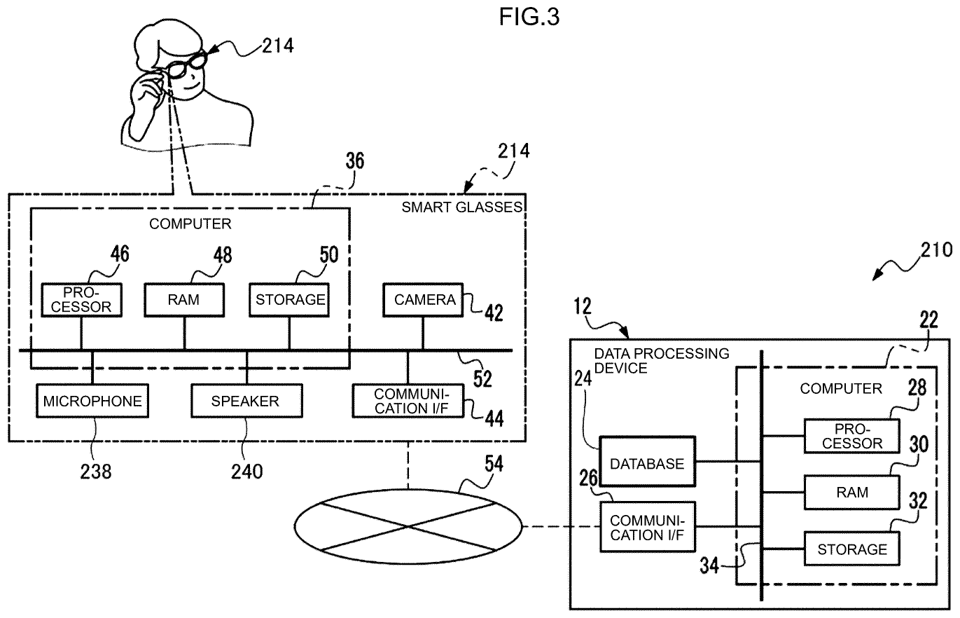

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

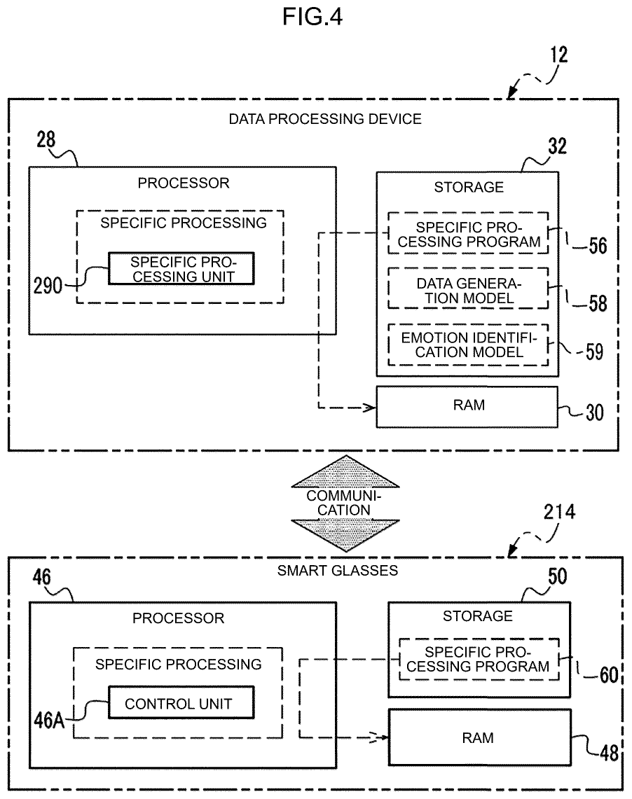

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, analysis unit, determination unit, and proposal unit is implemented by at least one of, for example, smart glasses 214 and a data processing device 12. For example, the reception unit is implemented by a microphone 238 of the smart glasses 214, allowing a patient to input symptoms by voice. The analysis unit is implemented, for example, by a specific processing unit 290 of the data processing device 12, and analyzes the input symptoms using a generative AI. The determination unit is implemented, for example, by the specific processing unit 290 of the data processing device 12, and determines the optimal consultation date and time based on the narrowed-down symptoms. The proposal unit is implemented, for example, by a control unit 46A of the smart glasses 214, and proposes an online diagnosis. The correspondence between each unit and the device or control unit is not limited to the examples described above and can be variously modified.

### Third Embodiment

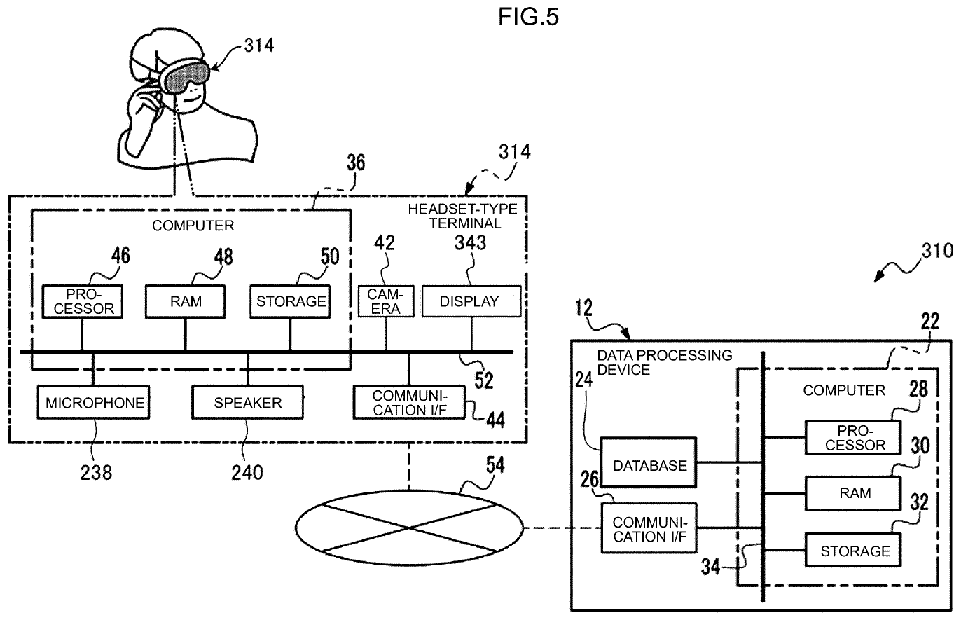

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

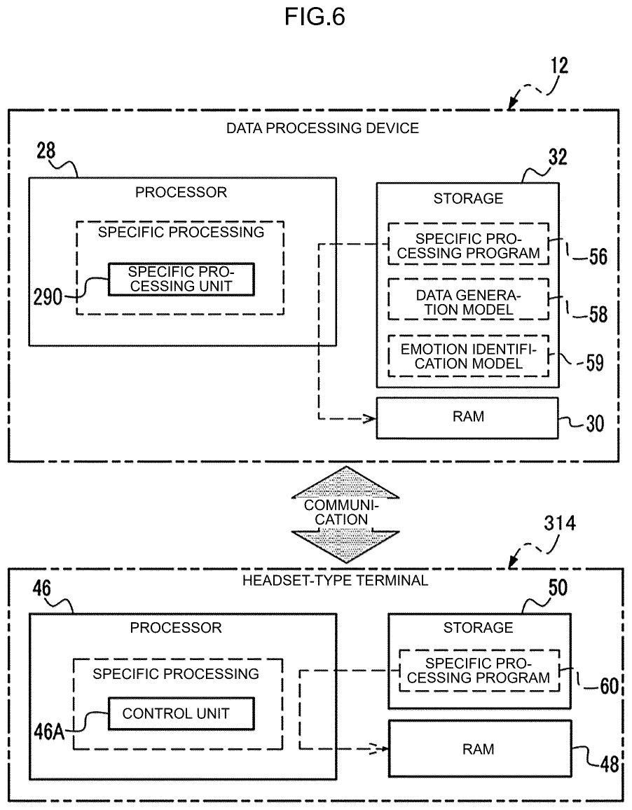

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, analysis unit, determination unit, and proposal unit is implemented by at least one of, for example, a headset-type terminal 314 and a data processing device 12. For example, the reception unit is implemented by a microphone 238 of the headset-type terminal 314, allowing a patient to input symptoms by voice. The analysis unit is implemented, for example, by a specific processing unit 290 of the data processing device 12, and analyzes the input symptoms using a generative AI. The determination unit is implemented, for example, by the specific processing unit 290 of the data processing device 12, and determines the optimal consultation date and time based on the narrowed-down symptoms. The proposal unit is implemented, for example, by a control unit 46A of the headset-type terminal 314, and proposes an online diagnosis. The correspondence between each unit and the device or control unit is not limited to the examples described above and can be variously modified.

### Fourth Embodiment

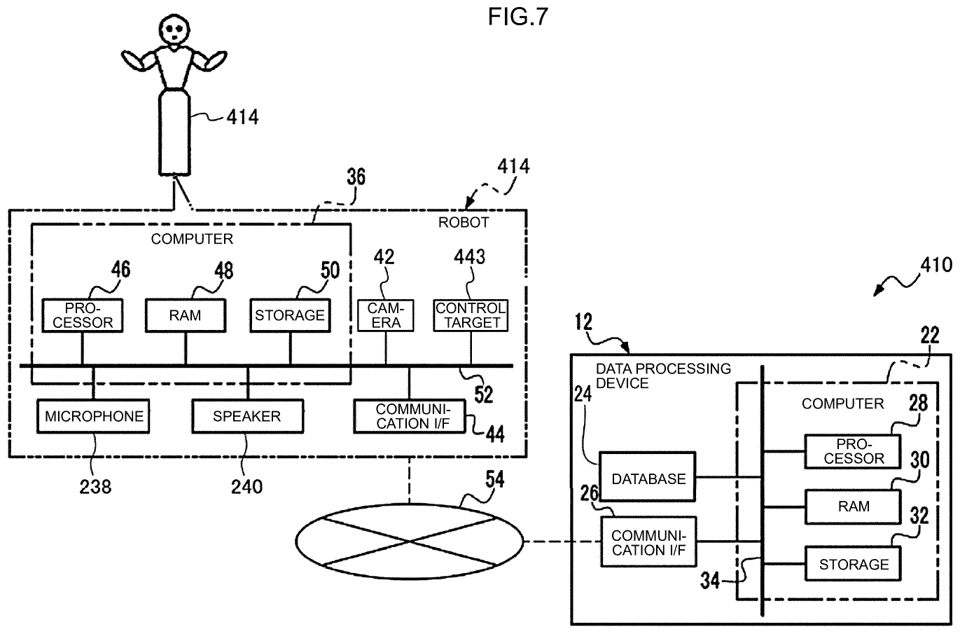

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

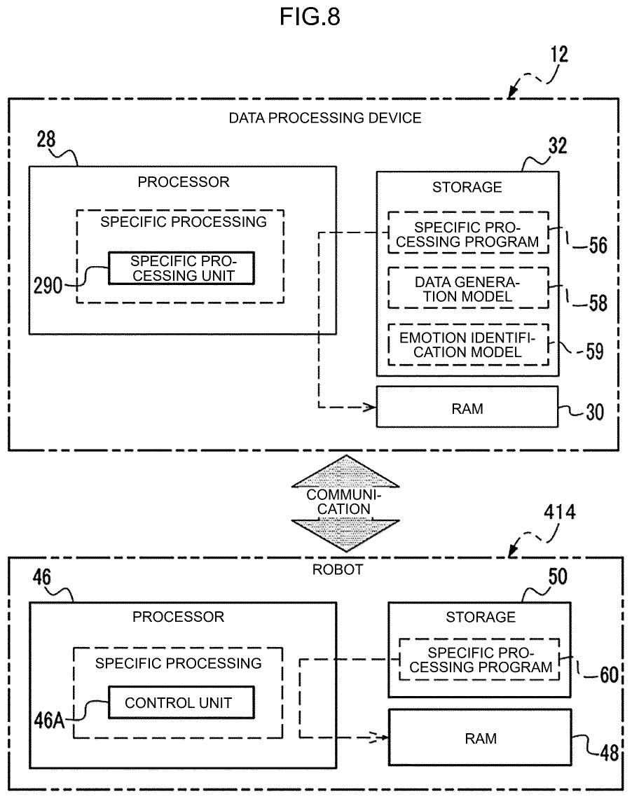

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, analysis unit, determination unit, and proposal unit is implemented by at least one of, for example, a robot 414 and a data processing device 12. For example, the reception unit is implemented by a microphone 238 of the robot 414, allowing a patient to input symptoms by voice. The analysis unit is implemented, for example, by a specific processing unit 290 of the data processing device 12, and analyzes the input symptoms using a generative AI. The determination unit is implemented, for example, by the specific processing unit 290 of the data processing device 12, and determines the optimal consultation date and time based on the narrowed-down symptoms. The proposal unit is implemented, for example, by a control unit 46A of the robot 414, and proposes an online diagnosis. The correspondence between each unit and the device or control unit is not limited to the examples described above and can be variously modified.

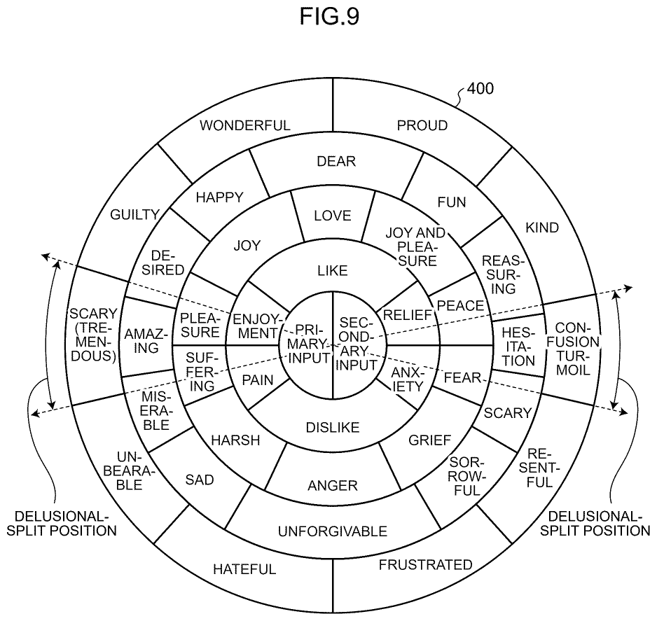

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

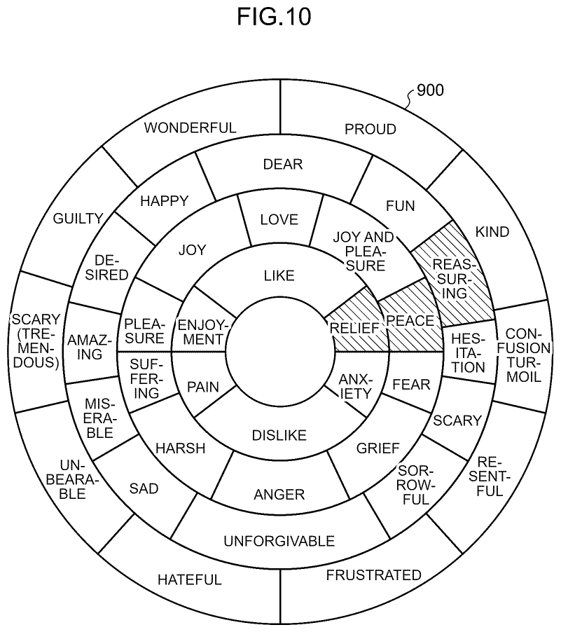

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a reception unit configured to input symptoms; an analysis unit configured to analyze the symptoms input by the reception unit; a determination unit configured to determine an optimal consultation date and time based on the symptoms analyzed by the analysis unit; and a proposal unit configured to propose an online diagnosis based on the consultation date and time determined by the determination unit.

(Supplementary Note 2) The system according to Supplementary Note 1, wherein the reception unit is configured to input symptoms of a patient.

(Supplementary Note 3) The system according to Supplementary Note 1, wherein the analysis unit is configured to analyze the symptoms of the patient by cooperation between a generative AI and an image analysis AI.

(Supplementary Note 4) The system according to Supplementary Note 1, wherein the determination unit is configured to determine an appropriate consultation date and time based on existing reservations and the desired date and time of the patient.

(Supplementary Note 5) The system according to Supplementary Note 1, wherein the proposal unit is configured to propose an online diagnosis based on a diagnosis result by the generative AI.

(Supplementary Note 6) The system according to Supplementary Note 1, wherein the analysis unit is configured to analyze the patient's complexion and facial expression by the image analysis AI and determine whether the symptoms are urgent.

(Supplementary Note 7) The system according to Supplementary Note 1, wherein the reception unit is configured to estimate the patient's emotions and adjust the symptom input interface based on the estimated emotions of the patient.

(Supplementary Note 8) The system according to Supplementary Note 1, wherein the reception unit is configured to analyze the patient's past consultation history and propose an appropriate symptom input method.

(Supplementary Note 9) The system according to Supplementary Note 1, wherein the reception unit is configured to filter input content based on the patient's current health condition and lifestyle habits when inputting symptoms.

(Supplementary Note 10) The system according to Supplementary Note 1, wherein the reception unit is configured to estimate the patient's emotions and determine the priority order of symptoms to be input based on the estimated emotions of the patient.

(Supplementary Note 11) The system according to Supplementary Note 1, wherein the reception unit is configured to prioritize input of highly relevant symptoms based on the patient's geographic location information when inputting symptoms.

(Supplementary Note 12) The system according to Supplementary Note 1, wherein the reception unit is configured to analyze the patient's social media activity and input relevant symptoms when inputting symptoms.

(Supplementary Note 13) The system according to Supplementary Note 1, wherein the analysis unit is configured to estimate the patient's emotions and adjust the accuracy of symptom analysis based on the estimated emotions of the patient.

(Supplementary Note 14) The system according to Supplementary Note 1, wherein the analysis unit is configured to optimize the analysis algorithm by referring to the patient's past consultation data when analyzing symptoms.

(Supplementary Note 15) The system according to Supplementary Note 1, wherein the analysis unit is configured to perform analysis based on the patient's living environment and occupational information when analyzing symptoms.

(Supplementary Note 16) The system according to Supplementary Note 1, wherein the analysis unit is configured to estimate the patient's emotions and adjust the display method of analysis results based on the estimated emotions of the patient.

(Supplementary Note 17) The system according to Supplementary Note 1, wherein the analysis unit is configured to perform analysis based on the patient's geographic distribution when analyzing symptoms.

(Supplementary Note 18) The system according to Supplementary Note 1, wherein the analysis unit is configured to improve the accuracy of analysis by referring to related literature of the patient when analyzing symptoms.

(Supplementary Note 19) The system according to Supplementary Note 1, wherein the determination unit is configured to estimate the patient's emotions and adjust the method of determining the consultation date and time based on the estimated emotions of the patient.

(Supplementary Note 20) The system according to Supplementary Note 1, wherein the determination unit is configured to select an optimal date and time by referring to the patient's past reservation history when determining the consultation date and time.

(Supplementary Note 21) The system according to Supplementary Note 1, wherein the determination unit is configured to determine the date and time based on the patient's current living situation and schedule when determining the consultation date and time.

(Supplementary Note 22) The system according to Supplementary Note 1, wherein the determination unit is configured to estimate the patient's emotions and determine the priority order of consultation dates and times based on the estimated emotions of the patient.

(Supplementary Note 23) The system according to Supplementary Note 1, wherein the determination unit is configured to select an optimal date and time by considering the patient's geographic location information when determining the consultation date and time.

(Supplementary Note 24) The system according to Supplementary Note 1, wherein the determination unit is configured to analyze the patient's social media activity and propose an optimal date and time when determining the consultation date and time.

(Supplementary Note 25) The system according to Supplementary Note 1, wherein the proposal unit is configured to estimate the patient's emotions and adjust the method of proposing online diagnosis based on the estimated emotions of the patient.

(Supplementary Note 26) The system according to Supplementary Note 1, wherein the proposal unit is configured to refer to the patient's past consultation data and make an optimal proposal when proposing online diagnosis.

(Supplementary Note 27) The system according to Supplementary Note 1, wherein the proposal unit is configured to make a proposal based on the patient's current health condition and lifestyle habits when proposing online diagnosis.

(Supplementary Note 28) The system according to Supplementary Note 1, wherein the proposal unit is configured to estimate the patient's emotions and determine the priority order of online diagnosis based on the estimated emotions of the patient.

(Supplementary Note 29) The system according to Supplementary Note 1, wherein the proposal unit is configured to make an appropriate proposal based on the patient's geographic location information when proposing online diagnosis.

(Supplementary Note 30) The system according to Supplementary Note 1, wherein the proposal unit is configured to analyze the patient's social media activity and make an optimal proposal when proposing online diagnosis.

What is claimed is:

1. A system comprising:
a communication interface configured to communicate with a client terminal via a packet-switched network;
a memory storing a data generation model obtained by deep learning on a neural network, and an emotion identification model; and
circuitry configured to:
receive, from the client terminal via the communication interface, input data comprising at least one of text data, voice data, or image data;
analyze the input data using a multimodal model comprising at least one of a convolutional neural network or a Transformer-based language model to generate classification data comprising at least one of a category label or a priority score;
estimate an emotion of a user by applying the emotion identification model to the input data;
generate, using the data generation model, scheduling data based on the classification data, the estimated emotion, and constraint data received from an external system via the communication interface;
generate, using the data generation model, remote service session data based on the classification data and the estimated emotion; and
transmit the scheduling data and the remote service session data to the client terminal via the communication interface and the packet-switched network.

2. The system according to claim 1, wherein the input data comprises symptom data of a patient, and wherein the classification data comprises at least one of an ICD-10 code classification or a severity score.

3. The system according to claim 1, wherein the multimodal model comprises a Transformer-based large language model configured to vectorize the text data using a multi-layer self-attention mechanism, and wherein the convolutional neural network comprises at least one of a ResNet architecture or an EfficientNet architecture configured to analyze image data.

4. The system according to claim 1, wherein the circuitry is further configured to preprocess the input data by performing at least one of noise removal, normalization, tokenization, or image resizing before analyzing the input data.

5. The system according to claim 1, wherein the priority score comprises an urgency probability value from 0 to 1, and wherein the circuitry is further configured to perform threshold determination based on the priority score.

6. The system according to claim 1, wherein the circuitry is further configured to adjust an input interface displayed on the client terminal based on the estimated emotion, such that when the estimated emotion indicates anxiety, the circuitry generates interface data for a simplified input interface, and when the estimated emotion indicates relaxation, the circuitry generates interface data for a detailed input interface.

7. The system according to claim 1, wherein the circuitry is further configured to analyze past input history associated with the user to generate candidate input data, and to transmit the candidate input data to the client terminal for display as selectable options.

8. The system according to claim 1, wherein the circuitry is further configured to receive attribute data of the user comprising at least one of a current health condition or a lifestyle habit, and to filter the classification data based on the attribute data.

9. The system according to claim 1, wherein the circuitry is further configured to determine a priority order of categories to be analyzed based on the estimated emotion, such that when the estimated emotion indicates anxiety, categories associated with high urgency are prioritized.

10. The system according to claim 1, wherein the circuitry is further configured to receive geographic location information from the client terminal and to adjust the classification data based on regional risk data associated with the geographic location information.

11. The system according to claim 1, wherein the circuitry is further configured to receive social media activity data of the user from the client terminal, analyze the social media activity data using a natural language processing model to extract status information, and adjust the classification data based on the extracted status information.

12. The system according to claim 1, wherein the circuitry is further configured to adjust an accuracy level of the analysis based on the estimated emotion, such that when the estimated emotion indicates anxiety, high-priority categories are analyzed with increased precision, and when the estimated emotion indicates relaxation, detailed analysis is performed across all categories.

13. The system according to claim 1, wherein the circuitry is further configured to optimize an analysis algorithm by referring to past classification data associated with the user stored in a database, and to extract time-series patterns from the past classification data.

14. The system according to claim 1, wherein the circuitry is further configured to receive environment data and occupation data of the user, and to adjust the classification data based on risk factors associated with the environment data and the occupation data.

15. The system according to claim 1, wherein the circuitry is further configured to adjust a display method of the classification data transmitted to the client terminal based on the estimated emotion, such that when the estimated emotion indicates anxiety, a simplified display format is used, and when the estimated emotion indicates relaxation, a detailed display format is used.

16. The system according to claim 1, wherein the circuitry is further configured to generate the scheduling data by cross-referencing the constraint data comprising availability data from the external system with desired time data received from the client terminal, and calculating an optimal time using a combinatorial optimization algorithm.

17. The system according to claim 1, wherein the circuitry is further configured to adjust a method of generating the scheduling data based on the estimated emotion, such that when the estimated emotion indicates anxiety, earlier available times are prioritized, and when the estimated emotion indicates urgency, immediate available times are searched.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model; and
circuitry configured to:
receive, from the client terminal via the communication interface, input data comprising at least one of text data input via the touch panel, voice data captured by the microphone, or image data captured by the camera;
preprocess the input data by performing at least one of noise removal, normalization, tokenization, or image resizing;
analyze the preprocessed input data using a multimodal model comprising a Transformer-based language model and a convolutional neural network to generate classification data comprising a category label and a priority score;
estimate an emotion of a user by applying the emotion identification model to the input data;
generate, using the data generation model, scheduling data based on the classification data, the estimated emotion, and constraint data received from an external system;
generate, using the data generation model, remote service session data based on the classification data and the estimated emotion;
adjust at least one of a format, a level of detail, or a display method of the scheduling data and the remote service session data based on the estimated emotion; and
transmit the scheduling data and the remote service session data to the client terminal via the communication interface, the scheduling data and the remote service session data causing the client terminal to present the scheduling data and the remote service session data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a data generation model obtained by deep learning on a neural network and an emotion identification model, the method comprising:
receiving, from a client terminal via the communication interface and a packet-switched network, input data comprising at least one of text data, voice data, or image data;
analyzing the input data using a multimodal model comprising at least one of a convolutional neural network or a Transformer-based language model to generate classification data comprising at least one of a category label or a priority score;
estimating an emotion of a user by applying the emotion identification model to the input data;
generating, using the data generation model, scheduling data based on the classification data, the estimated emotion, and constraint data received from an external system via the communication interface;
generating, using the data generation model, remote service session data based on the classification data and the estimated emotion; and
transmitting the scheduling data and the remote service session data to the client terminal via the communication interface and the packet-switched network.
